SEQUENCE LISTING (5 sequences)

SEQ ID NO: 1 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 2 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 3 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 4 - protein, 976 aa, Homo sapiens [sequence omitted]

SEQ ID NO: 5 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

(19) United States
(12) Patent Application Publication
(10) Pub. No.: US 2009/0306207 A1
(43) Pub. Date: Dec. 10, 2009

(54) Treatment of Drug-Resistant Proliferative Disorders

(76) Inventors: M. V. Ramana Reddy, Upper Darby, PA (US); E. Premkumar Reddy, Villanova, PA (US); Stephen C. Cosenza, Voorhees, NJ (US); Stacey J. Baker, Huntingdon Valley, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/794,524

(22) PCT Filed: Jan. 4, 2006
(86) PCT No.: PCT/US06/00059

§ 371 Date: Jun. 28, 2007

Related U.S. Application Data
(60) Provisional application No. 60/641,378, filed on Jan. 5, 2005.

(51) Int. Cl. A61K 31/10; A61P 35/00; A61K 31/196

(52) U.S. Cl. 514/562; 514/710

(57) ABSTRACT

α,β-Unsaturated sulfones, sulfoxides and sulfonamides according to Formula I:

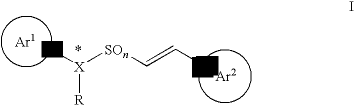

wherein Ar¹, Ar², X, n, * and R are as defined herein are useful for the treatment of proliferative disorders which are resistant to treatment by ATP-competitive kinase inhibitors.

### FIELD OF THE INVENTION

The invention relates to the treatment of proliferative disorders that are resistant to therapeutic agents that are ATP-competitive kinase inhibitors.

### BACKGROUND OF THE INVENTION

### I. Resistance of Proliferative Disorders to ATP-Competitive Kinase Inhibitors

Protein kinases have been shown to regulate cell proliferation. Inhibition of protein kinases has emerged as a research area that holds potential for development of new treatments for proliferative disorders, particularly cancer.

Inhibition of protein kinases has been accomplished therapeutically by administration of ATP-competitive small molecules. Such inhibitors block the enzymatic activity of kinases and thereby interfere with phosphorylation of cellular substrates. Examples of ATP-competitive small molecule inhibitors of kinases are shown in Table 1.

ATP-competitive kinase inhibitors have been shown to create selective pressures in target proliferating cells associated with the disorders for which the inhibitors are therapeutically administered. These selective pressures often result in the development of resistance in the target cells. Resistance may arise from mutation of the targeted kinase.

TABLE 1
ATP-competitive small molecule inhibitors of kinases

| Compound Name | Compound Structure |
|---|---|
| PD180970 | 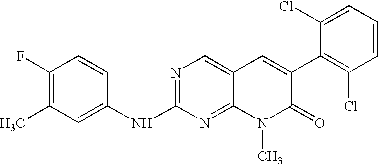 |
| BMS-354825 | 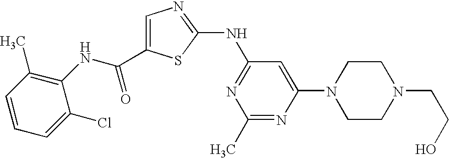 |
| Imatinib | 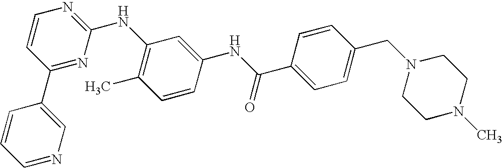 |
| SU5416 | 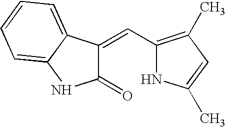 |
| SU6668 | 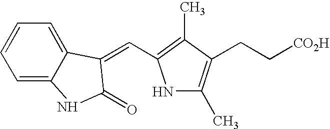 |
| SU11248 | 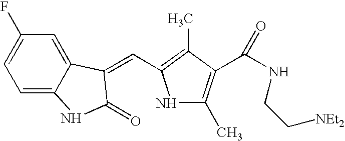 |
| AP23464 | 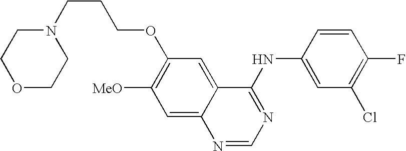 |
| Gefitinib | 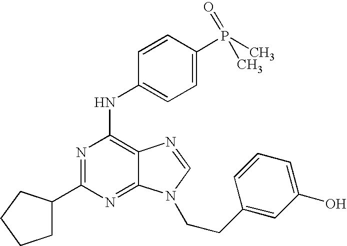 |
| Erlotinib | 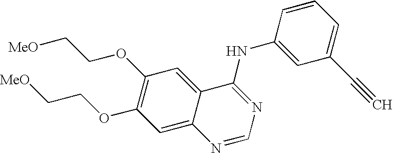 |
| PD153035 | 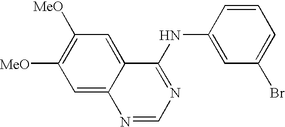 |
| SB 203580 | 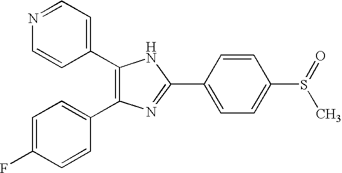 |

### A. BCR-ABL Associated Proliferative Disorders

1. Molecular Basis for the Disorder

Chronic myelogenous leukemia (CML), often referred to as chronic myeloid leukemia, was the first neoplastic disease to be associated with a specific chromosomal abnormality, namely the Philadelphia or Ph¹ chromosome. At the molecular level, the most notable feature is the translocation of the proto-oncogene c-abl from the long arm of chromosome 9 to the breakpoint cluster region (bcr) on chromosome 22, resulting in the formation of bcr-abl hybrid genes. The c-abl proto-oncogene normally encodes a protein (ABL) having tyrosine kinase activity. In cells carrying bcr-abl hybrid genes, the oncogene BCR-ABL is expressed. The tyrosine kinase activity of BCR-ABL is substantially augmented compared to the tyrosine kinase activity of ABL.

The BCR-ABL fusion protein commonly found in Philadelphia chromosome CML patients is a protein of approximately 210 kilodaltons (p210 wt or p210BCR-ABL). The fusion transcript typically results from BCR exon 13 or 14 joined to ABL exon 2. (Chissoe et al (1995) Genomics 27:67-82). The ABL portion of the p210wt therefore consists of exons 2-11 of the c-ABL gene. The common convention in the literature to identify positions in the ABL tyrosine kinase portion in a particular BCR-ABL allele is to use the amino acid numbering for c-ABL which results from alternative splicing using exon 1a (protein ID AAB60394.1 and GenBank Accession number U07563). Therefore the mutations cited within this specification are likewise keyed to the amino acid numbering of this splice variant whose sequence is provided as SEQ ID NO: 1. The first 26 amino acids of SEQ ID NO: 1 are encoded by ABL exon 1a and are not found in the BCR-ABL fusion protein p210 wt. The amino acid sequence encoded by exon 2 starts with E27 of proto-oncogene ABL which results from the transcript which results form the alternative splicing that uses exon 1a. The protein kinase domain is from approximately Ile 242 to amino acid 493. In this numbering system, the phosphate-binding loop (p-loop) is amino acids 249-256, the catalytic region is amino acids 361-367 and the activation loop is amino acids 380-402.

The ATP-competitive tyrosine kinase inhibitor imatinib is highly effective in inhibiting the tyrosine kinases ABL, KIT and platelet derived growth factor receptor (PDGFR). Therapeutic administration of imatinib demonstrates clinical efficacy in the treatment of CML and acute lymphoblastic lymphoma (ALL). BCR-ABL is also implicated in idiopathic pulmonary fibrosis, wherein inhibition of ABL by imatinib has been shown to prevent cell proliferation. Daniels et al., J. Clin. Invest., 114, 1308-16 (2004). However, a portion of imatinib-treated patients treated relapse when imatinib-resistant cells emerge.

2. Mutation of BCR-ABL Confers Imatinib Resistance

Imatinib resistance may result from kinase mutations at the site of imatinib binding. Mutations may occur in the kinase catalytic domain, which includes the so-called ‘gatekeeper’ residue, Thr315, and other residues that contact imatinib during binding (e.g., Phe317 and Phe 359). Other mutations occur in the ATP binding domain (the p-loop) and in the activation loop, an area of the kinase structure involved in a conformational change that occurs upon imatinib binding.

Imatinib binding to BCR-ABL involves formation of a hydrogen bond to the Thr315 hydroxyl group. The substitution of isoleucine for threonine at position 315 is one of the most frequent BCR-ABL mutations in imatinib-resistant CML. Alteration of Thr315 to the larger and non-hydrogen bonding isoleucine directly interferes with imatinib binding. Thr315, though necessary for binding of imatinib, is not required for ATP binding to BCR-ABL. Thus, the catalytic activity, and therefore the tumor-promoting function of the BCR-ABL oncoprotein, is preserved in the T315I mutant.

Imatinib binding is also affected by mutations in the kinase p-loop. BCR-ABL sequence analysis in relapsed CML and Ph+ ALL patients has detected p-loop mutations at Tyr253 and Glu255. Amino-acid substitutions at these positions may interfere with the distorted p-loop conformation required for imatinib binding. This is consistent with the observed in vivo resistance and the particularly poor prognosis of patients affected by BCR-ABL p-loop mutations such as Y253F and E255K (Branford et al., Blood, 102, 276-283 (2003).

Imatinib binding is associated with the inactive, unphosphorylated state of the BCR-ABL activation loop. Mutations in this region, such as H396R, destabilize a closed conformation of the activation loop and thereby counteract imatinib inhibition.

Another group of point mutations, remote from the imatinib binding site, lie in the carboxy-terminal lobe of the kinase domain. The most frequently detected BCR-ABL mutation falling into this group is M351T. The M351T mutation accounts for 15-20% of all cases of imatinib clinical resistance (Hochhaus et al., Leukemia, 18, 1321-31 (2004). M351T mutation appears to affect the precise positioning of residues in direct contact with imatinib (Cowan-Jacob et al., Mini Rev. Med. Chem., 4, 285-299 (2004), and Shah et al, Cancer Cell, 2, 117-125 (2002).

The above BCR-ABL mutations account for most of the BCR-ABL mutations which have been associated with imatinib resistance. However, a saturating mutational analysis of full-length BCR-ABL combined with a cellular screening procedure selecting for BCR-ABL-driven cell proliferation in the presence of imatinib, has revealed that mutations outside of the kinase domain can weaken kinase interaction with imatinib and thereby contribute to target resistance (Azam, et al., Cell, 112, 831-843 (2003).

BCR-ABL mutations clinically relevant to development of resistance to ATP-competitive kinase inhibitors such as imatinib include the following: G250E, F317L, Y253F, H396R, F311L, M351T, T315, H396P, E255V, Y253H, Q252H, M244V, L387M, E355G, E255K and F359V. See, von Bubnoff et al., Leukemia 17, 829-838 (2003); Cowan-Jacob, et al., Mini Rev. Med. Chem. 4, 285-299 (2004); Hochhaus, et al., Leukemia 18, 1321-1331 (2004); Nardi, et al., Curr. Opin. Hematol. 11, 3543 (2004); Ross, et al., Br. J. Cancer 90, 12-19; and Daub et al., Nature Reviews Drug Discovery, 3, 1001-10 (2004).

The therapeutic agents PD180970, BMS-354825 and AP23464 have demonstrated effectiveness against some imatinib-resistant proliferative disorders (Daub et al., supra). However, no agent has shown effectiveness against all BCR-ABL mutations. Furthermore, no agent is effective against resistance conferred by the T315I mutation.

### B. Platelet Derived Growth Factor (PDGF) Associated Proliferative Disorders

Platelet-derived growth factor receptor (PDGFR) is another tyrosine kinase. Molecular mechanisms, similar to the chromosomal translocation that generates the BCR-ABL oncoprotein, have been shown to create the imatinib-sensitive PDGFR fusion proteins PDGFRα (SEQ ID NO: 2) and PDGFRβ (SEQ ID NO: 3). PDGFRα and PDGFRβ are implicated in idiopathic hypereosinophilic syndrome and chronic myelomonocytic leukemia, respectively (Apperley et al., N. Engl. J. Med., 347, 481-487 (2002), and Cools et al., N. Engl. J. Med, 348, 1201-14 (2003). In hypereosinophilic syndrome, excessive proliferation of eosinophils often results from the constitutive tyrosine kinase activity of the chimeric Fip1-like (FIP1L1)-PDGFR protein, which arises from the fusion of the FIP1L1 and PDGFRα.

PDGFR and the proto-oncogene c-kit are implicated in malignant fibrous histocytoma (MFH). Imatinib has demonstrated inhibition of cell proliferation in three MFH cell lines (TNMY1, GBS-1 and Nara-F) that express one or both of the target kinases (Kawamoto et al., Anticancer Res., 24, 2675-9 (2004)).

PDGFRβ has also been implicated in prostate cancers, (Hofer et al., Neoplasia, 6, 503-12 (2004)), androgen dependent prostate cancers (Mathew et al., J. Clin. Oncol., 22, 3323-9 (2004)) and in dermatofibrosarcoma (Klener et al., Cas. Lek. Cesk., 143, 582-3 (2004)).

PDGFR and BCR-ABL are also implicated in endometrioid endometrial carcinoma (EEC) and uterine papillary serous carcinoma (UPSC) (Slomovitz et al., Gyneco. Oncol., 95, 32-36 (2004)).

PDGFRβ has also been implicated in chordoma, and clinical administration of imatinib has been shown to have antitumor activity in a group of chordoma patients (Casali et al., Cancer, 1, 2086-97 (2004)).

PDGFRα and PDGFRβ are expressed in virtually all glioma cell lines and in fresh surgical isolates of human malignant astrocytoma. Imatinib has been shown to inhibit the growth of human glioblastoma cells (U343 and U87) in vivo in a nude mouse (Kilic et al., Cancer Res., 60, 5143-50 (2000)).

In one study, five out of five imatinib-treated patients with a detectable FIP1L1-PDGFR gene fusion experienced complete hematological remission (Cools et al., Id). One of the five patients eventually relapsed and was found to be resistant to imatinib. The imatinib resistance was attributed to a point mutation in the αPDGFR αATP binding site. The threonine-to-isoleucine substitution (T674I) occurs at a position equivalent to the T315I mutation of the gatekeeper residue of BCR-ABL.

The gatekeeper residue of BCR-ABL corresponds to gatekeeper residues on several kinases, including PDGFR, KIT, EGFR, SRC and P38. Despite clinical advances in the treatment of kinase-associated disorders, clinically relevant mutations, have been identified in the kinase domains of PDGFR, KIT, and EGFR. Several of these mutations correspond to mutations that occur in BCR-ABL, and include gatekeeper residues. Mutation of the gatekeeper residue to a larger residue, such as isoleucine or methionine, has been shown to confer resistance in the kinases p38, SRC and EGFR to ATP-competitive inhibitors SB203580, PP1 and PD153035, respectively (Eyers et al., Chem. Biol., 5, 321-328 (1998); Liu et al., Chem. Biol., 8, 257-266 (1999); and Blencke et al., J. Biol. Chem., 278, 15435-40 (2003).

### C. KIT Associated Proliferative Disorders

Gastrointestinal stromal tumors (GIST), which are mesenchymal tumors of the stomach and small intestine, express the KIT tyrosine kinase receptor (SEQ ID NO: 4). GIST cells often contain an activating point mutation in the KIT kinase domain which leads to a constitutive kinase. Expression of activating KIT mutations in mice has been shown to cause tumors similar to human GISTs (Sommer et al., Proc. Natl. Acad. Sci., 100, 6706-11 (2003). C-kit expression has also been implicated in medulloblastoma, a highly invasive primitive neuroectodermal tumor of the cerebellum, which is the most common childhood malignant central nervous system tumor. In a study of ten medulloblastoma tumor samples, all expressed c-kit (Chilton-Macneill et al., Pediatr. Dev. Pathol., 7, 493-8 (2004)). C-kit has also been shown to be expressed in many uterine leiomyosarcomas (Raspollini et al., Clin. Cancer Res., 10, 3500-3 (2004)).

Secondary resistance formation to imatinib has been reported in at least one GIST patient. The resistance was caused by a T670I mutation in the KIT kinase domain; the mutation site corresponds to the T315I gatekeeper residue of BCR-ABL (Demitri et al., N. Engl. J. Med, 347, 472-480 (2002). The mutation was confined to a metastatic lesion of the solid tumor, which progressed during continued imatinib therapy while other lesions were still responding to imatinib treatment (Tamborini et al., Gastroenterology, 127, 294-299, (2004).

Another secondary KIT mutation, Y823D, has been demonstrated to emerge during imatinib therapy and confer significant imatinib resistance. This point mutation is at a position that corresponds to the major phosphorylation site Tyr393 within the ABL activation loop (Wakai et al., Br. J. Cancer, 90, 2059-61 (2004). Phosphorylation of this site in wild-type ABL serves to destabilize the inactive conformation of the activation loop (Schindler et al., Science, 289, 1938-42 (2000). It has been suggested that the Y823D KIT mutation may mimic the phosphorylated Tyr393 of ABL, thereby conferring similar imatinib resistance (Daub et al., Nature Reviews Drug Discovery, 3, 1001-10 (2004)).

Another mutation has been characterized in the KIT activation loop. The kinase-activating D816V mutation in the KIT protein confers primary imatinib resistance and has been identified as a cause of disease in human mastocytosis (Ma et al., Blood, 99, 2059-61 (2002).

### D. EGFR Associated Proliferative Disorders

Epidermal growth factor receptor (EGFR, SEQ ID NO: 5) is another tyrosine kinase. Gefitinib, an EGFR inhibitor, is approved for the treatment of advanced non-small-cell lung cancers (NSCLCs) that do not respond to established chemotherapy regimens (Muhsin et al., Nature Rev. Drug Discov., 2, 515-516 (2003) and Cohen et al., Clin. Cancer Res., 10, 1212-18 (2004). The C-to-T single-nucleotide mutation that leads to the imatinib-refractory T315I BCR-ABL mutant also dramatically desensitizes EGFR to gefitinib by replacing the corresponding gatekeeper residue Thr766 with a methionine residue (T766M) (Blenke et al., Chem. Biol., 11, 691-791 (2004), and Blenke et al., J. Biol. Chem, 278, 15435-40 (2003).

### II. Prior Attempts To Overcome Imatinib Resistance

Pyrido[2,3-d]pyrimidines such as PD180970 (Table 1) have been shown to suppress the cancer-promoting activity of BCR-ABL activation loop mutants such as H396P (La Rosée et al., Cancer Res., 62, 7149-53 (2002), and von Bubnoff et al., Cancer Res., 9, 1267-73 (2003)). These compounds also inhibit BCR-ABL which contains the clinically common p-loop mutations associated with Tyr253 or Glu255. PD180970 has also been shown to inhibit BCR-ABL which contains the M351T mutant, which mutation accounts for about 15-20% of imatinib-resistant CML cases (Kantarjian et al., Blood, 101, 473-475 (2003)).

SRC, the first proto-oncogenic protein described, is a non-receptor tyrosine kinase. The SRC and ABL inhibitors BMS-354825 and AP23464 (Table 1) have shown activity similar to pyrido[2,3-d]pyrimidines in inhibiting BCR-ABL containing certain clinically common mutations associated with imatinib resistance (Shah et al., Science, 305, 399-401 (2004) and O'Hare et al., Blood, 104, 2532-39 (2004).

### III. α,β-Unsaturated Sulfones, Sulfoxides and Sulfonamides

Certain α,β-unsaturated sulfones, sulfoxides and sulfonamides, particularly styrylbenzyl sulfones, styrylbenzyl sulfoxides and styryl sulfonamides have been shown to possess antiproliferative, radioprotective and chemoprotective activity. See: U.S. Pat. Nos. 6,599,932, 6,576,675, 6,548,553, 6,541,475, 6,486,210, 6,414,034, 6,359,013, 6,201,154, 6,656,973 and 6,762,207. However, none of these α,β-unsaturated sulfones, sulfoxides and sulfonamides have been reported to have activity in the treatment of kinase-dependent proliferative disorders that are resistant to treatment with ATP-competitive kinase inhibitors.

Some advances have been made in overcoming drug resistance caused by kinase mutations. However, new therapies are needed which are capable of treating proliferative disorders, particularly cancers, that are resistant to treatment by ATP-competitive kinase inhibitors and capable of preventing the development of such resistance. In addition, new therapies are needed which are capable of preventing or delaying the emergence of proliferative disorders, particularly cancers, that are resistant to treatment by ATP-competitive kinase inhibitors.

### SUMMARY OF THE INVENTION

According to one embodiment of the invention, there is provided a method of treating a kinase-dependent proliferative disorder in an individual, particularly a cancer, that is resistant to treatment with an ATP-competitive kinase inhibitor. The method comprises administering to the individual in need of such treatment an effective amount of at least one compound according to Formula I:

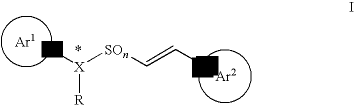

wherein:

Ar¹ and Ar² are independently selected from substituted and unsubstituted aryl and substituted and unsubstituted heteroaryl;

X is N or CH;

n is 1 or 2;

R is —H or —(C₁-C₈)hydrocarbyl; and

* indicates that, when X is CH, and R is other than —H, the conformation of the substituents on the carbon atom of X is R—, S— or any mixture of R— and S—; or a salt thereof;

provided that when X is N, then n is 2.

According to another embodiment of the invention, a method is provided of preventing or delaying, in an individual suffering from a kinase-dependent proliferative disorder, the development of resistance to therapy which includes administration of an ATP-competitive kinase inhibitor. The method comprises administering to the individual in need of such treatment an effective amount of at least one compound according to Formula I, as defined above. According to one embodiment, the method father comprising administering an effective amount of at least one ATP-competitive kinase inhibitor.

The resistance of the kinase-dependent proliferative disorder to treatment with a ATP-competitive kinase inhibitor results from a mutation in the protein sequence of the kinase associated with the kinase-dependent proliferative disorder.

According to one embodiment of the invention, the kinase-dependent proliferative disorder that is treated is resistant to at least one ATP-competitive BCR-ABL inhibitor.

According to one embodiment of the invention, the resistance to an ATP-competitive inhibitor of BCR-ABL results from a mutation of one or more amino acid residues within a kinase domain of the BCR-ABL protein. According to some embodiments, the resistance arises from a mutation of at least one residue within the BCR-ABL p-loop. According to particular embodiments, the mutation comprises an alteration of BCR-ABL Tyr 253 or Glu255. According to other embodiments, the resistance arises from a mutation of at least one residue within the BCR-ABL activation loop. According to particular embodiments, the mutation is an alteration of His396.

According to a preferred embodiment, the ATP-competitive BCR-ABL inhibitor is imatinib.

According to other embodiments, the resistance to an ATP-competitive inhibitor of BCR-ABL results from a mutation within the BCR-ABL protein comprising at least one mutation selected from the group consisting of F317L, H396R, M351T, H396P, Y253H, M244V, E355G, F359Y, G250E, Y253F, F311L, T315I, E255V, Q252H, L387M, E255K.

According to some embodiments of the invention, the kinase-dependent proliferative disorder that is resistant to treatment with an ATP-competitive kinase inhibitor is selected from the group consisting of chronic myelogenous leukemia, acute lymphoblastic lymphoma, idiopathic pulmonary fibrosis, idiopathic hypereosinophilic syndrome, chronic myelomonocytic leukemia, malignant fibrous histiocytoma, prostate cancers, androgen dependent prostate cancers, dermatofibrosarcoma, endometrioid endometrial carcinoma, uterine papillary serous carcinoma, chordoma, glioma, malignant astrocytoma, glioblastoma, gastrointestinal stromal tumors, medulloblastoma, uterine leiomyosarcomas, and non-small-cell lung cancer.

According to one embodiment of the invention, the kinase-dependent proliferative disorder is resistant to at least one ATP-competitive inhibitor of KIT, which resistance results from mutation of one or more amino acid residues within the KIT protein. According to one embodiment, the mutation is within the KIT kinase domain.

According to certain embodiments, the mutation within the KIT protein comprises an alteration of at least one of Thr670, Tyr823 or Asp816. According to other embodiments, the mutation within the KIT protein comprises at least one mutation selected from the group consisting of T670I, Y823D and D816V.

According to one embodiment of the invention, the kinase-dependent proliferative disorder is resistant to at least one ATP-competitive inhibitor of EGFR, which resistance results from mutation of one or more amino acid residues within the EGFR protein. According to one embodiment, the mutation is within the EGFR kinase domain.

According to certain embodiments, the EGFR mutation comprises an alteration of Thr766. One such mutation is the mutation T766M.

According to another embodiment of the invention, the kinase-dependent proliferative disorder is resistant to at least one inhibitor of PDGFRα, which resistance results from mutation of one or more amino acid residues within the PDGFRα protein. According to one embodiment, the mutation is within the PDGFRα kinase domain.

According to certain embodiments, the PDGFRα mutation comprises an alteration of Thr674. One such mutation is the mutation is T674I.

According to another embodiment of the invention, the kinase-dependent proliferative disorder is resistant to at least one inhibitor of PDGFRβ, which resistance results from mutation of one or more amino acid residues within the PDGFRβ protein. According to one embodiment, the mutation is within the PDGFRα kinase domain.

According to certain embodiments, the PDGFRβ mutation comprises an alteration of Thr681. One such mutation is the mutation is T681I.

According to an embodiment of the invention, substituents on substituted aryl or heteroaryl Ar¹ in Formula I may be independently selected from the group consisting of halogen, —(C₁-C₈)hydrocarbyl, —(═O)R², —NR²₂, —NHC(═O)R³, —NHSO₂R³, —NH(C₂-C₆)alkylene-C(═O)R⁶, —NHCR²R⁴C(═O)R⁶, —C(═O)OR², —C(═O)NR²₂, —NO₂, —CN, —OR², —OC(═O)R³, —OSO₂R³, —O(C₂-C₆)alkylene-C(═O)R⁶, —OCR²R⁴C(═O)R⁶, —P(═O)(OR²)₂, —OP(═O)(OR²)₂, —O(C₂-C₆ alkylene)N(C₁-C₃)alkyl)₂, —NHC(═NH)NHR², —(C₁-C₆)haloalkyl, —O(C₁-C₆)haloalkyl; —SO₂NH₂; and —N═CH—R⁷; and substituents on substituted aryl or heteroaryl Ar² in Formula I may be independently selected from the group consisting of —(C₁-C₈)hydrocarbyl, —C(═O)R², halogen, —NO₂, —CN, —OR², —C(═O)OR², —NR²₂, —(C₁-C₆)haloalkyl; —SO₂NH₂; and —O(C₁-C₆)haloalkyl;

wherein:

each R² is independently selected from the group consisting of —H and —(C₁-C₈)hydrocarbyl;

each R³ is independently selected from the group consisting of —(C₁-C₈)hydrocarbyl, —O(C₁-C₈)hydrocarbyl, substituted and unsubstituted aryl, substituted and unsubstituted heterocyclyl(C₁-C₃)alkyl, substituted and unsubstituted heteroaryl(C₁-C₃)alkyl, —(C₂-C₁₀)heteroalkyl, —(C₁-C₆)haloalkyl, —CR²R⁴NHR⁵, —NR²₂, —C₁-C₃)alkyleneNH₂, —C₁-C₃)alkylene-N((C₁-C₃)alkyl)₂, —(C₁-C₃)perfluoroalkylene-N((C₁-C₃)alkyl)₂, —(C₁-C₃)alkylene-N⁺((C₁-C₃)alkyl)₃, —C₁-C₃)alkylene-N⁺(CH₂CH₂OH)₃, —(C₁-C₃)alkylene-OR², —(C₁-C₄)alkylene-CO₂R², —(C₁-C₄)alkylene-C(═O)halogen, halo(C₁-C₃)alkyl-, —(C₁-C₃)alkylene-C(═O)(C₁-C₃)alkyl, and —(C₁-C₄)perfluoroalkylene-CO₂R²;

each R⁴ is independently selected from the group consisting of —H, —(C₁-C₆)alkyl, —(CH₂)₃—NH—C(NH₂)(═NH), —CH₂C(═O)NH₂, —CH₂CO₂R², —CH₂SH, —(CH₂)₂C(═O)—NH₂, —(CH₂)₂CO₂R², —CH₂-(2-imidazolyl), —(CH₂)₄—NH₂, —(CH₂)₂—S—CH₃, phenyl, —CH₂-phenyl, —CH₂—OH, —CH(OH)—CH₃, —CH₂-(3-indolyl), and —CH₂-(4-hydroxyphenyl);

each R⁵ is independently selected from the group consisting of —H, —C(═O)(C₁-C₇)hydrocarbyl and a carboxy terminally-linked peptidyl residue containing from 1 to 3 amino acids in which the terminal amino group of the peptidyl residue is present as a functional group selected from the group consisting of —NH₂, —NHC(═O)(C₁-C₆)alkyl, —NH(C₁-C₆)alkyl, —N(C₁-C₆alkyl)₂ and —NHC(═O)O(C₁-C₇)hydrocarbyl;

each R⁶ is independently selected from the group consisting of —OR², —NR²₂, and an amino terminally-linked peptidyl residue containing from 1 to 3 amino acids in which the terminal carboxyl group of the peptidyl residue is present as a functional group selected from the group consisting of —CO₂R² and —(═O)NR²₂; and

each R⁷ is independently selected from the group consisting of substituted and unsubstituted aryl and substituted and unsubstituted heteroaryl;

provided that the highest number of substituents on Ar¹ and Ar² is equal to the number of substitutable hydrogen atoms in the ring to which the substituents are attached.

Substituents on substituted aryl or heteroaryl groups that comprise R³ or R⁷ are preferably independently selected from the group consisting of —(C₁-C₆)alkyl, —(C₁-C₆)alkoxy, halogen, —C(═O)(C₁-C₆)alkyl, —NH₂, —NH(C₁-C₆)alkyl, —N(C₁-C₆)alkyl)₂, —NHC(═O)(C₁-C₆)alkyl, —NO₂, —CN, (C₁-C₆)haloalkyl, —(C₁-C₆)alkylene-NH₂, —CO₂H, CONH₂, C(═N)NH₂, and heterocyclyl(C₁-C₆)alkyl; wherein heterocyclyl rings comprising heterocyclyl(C₁-C₆)alkyl are optionally substituted by —(C₁-C₆)alkyl or C(═O)(C₁-C₆)alkyl.

Substituents on substituted heterocyclyl groups that comprise R³ are preferably independently selected from the group consisting of —(C₁-C₆)alkyl, —(C₁-C₆)alkoxy, halogen, —C(═O)(C₁-C₆)alkyl, —CO₂H, and CONH₂.

According to some preferred embodiments of the invention, Ar¹ is phenyl. According to other preferred embodiments of the invention, Ar² is phenyl. According to still other preferred embodiments of the invention, both Ar¹ and Ar² are phenyl.

Preferably, when Ar¹ and Ar² are both phenyl, both Ar¹ and Ar² are at least mono-substituted.

According to some embodiments of the invention, the aryl and heteroaryl groups comprising Ar¹ and Ar² in Formula I compounds are mono-, di- or tri-substituted. According to other embodiments, the aryl and heteroaryl groups comprising Ar¹ and Ar² are substituted at all substitutable positions.

R is preferably —H or —(C₁-C₈)alkyl, most preferably —H or —(C₁-C₆)alkyl.

According to a preferred embodiment, substituents on substituted aryl or heteroaryl Ar¹ are independently selected from the group consisting of halogen, —NR²₂, —NHCR²R⁴C(═O)R⁶, —OR², —OCR²R⁴C(═O)R⁶, and —OP(═O)(OR²)₂; and substituents on substituted aryl or heteroaryl Ar² are independently selected from the group consisting of (C₁-C₈)hydrocarbyl, halogen, —OR², —C(═O)OR², and —NR²₂.

According to a more preferred embodiment, substituents on substituted aryl or heteroaryl Ar¹ are independently selected from the group consisting of halogen, —NH₂, —NHCHR⁴C(═O)OR², —OH, —OCHR⁴C(═O)OR², and —OP(═O)(OR²)₂; and substituents on substituted aryl or heteroaryl Ar² are independently selected from the group consisting of (C₁-C₆)alkyl, halogen, —OR², —C(═O)OR², and —NR²₂.

According to a most preferred embodiment, substituents on substituted aryl or heteroaryl Ar¹ are independently selected from the group consisting of halogen, —NH₂, —NHCH(CH₃)C(═O)OH, —NHCH₂C(═O)OH, —OH, —OCH(CH₃)C(—O)OH, and —OCH₂C(═O)OH; and substituents on substituted aryl or heteroaryl Ar² are independently selected from the group consisting of —(C₁-C₃)alkyl, halogen, —O(C₁-C₆)alkyl, —C(═O)OR², and —NR²₂.

Preferred compounds according to Formula I include, for example: (E)-2-(5-((2,4,6-trimethoxystyrylsulfonyl)methyl)-2-methoxyphenylamino)acetic acid; (racemic)-(E)-2-(5-((2,4,6-trimethoxystyrylsulfonyl)methyl)-2-methoxyphenylamino)propanoic acid; (R)-(E)-2-(5-((2,4,6-trimethoxystyrylsulfonyl)methyl)-2-methoxyphenylamino)propanoic acid; (S)-(E)-2-(5-((2,4,6-trimethoxystyrylsulfonyl)methyl)-2-methoxyphenylamino)propanoic acid; (E)-2-(5-((2,4,6-trimethoxystyrylsulfinyl)methyl)-2-methoxyphenylamino)acetic acid; (E)-2-(5-((2,4,6-trimethoxystyrylsulfinyl)methyl)-2-methoxyphenylamino)propanoic acid; (E)-2,4,6-trimethoxystyryl-N-(3-carboxymethylamino-4-methoxy-phenyl)sulfonamide; (S)-(E)-2-(5-((2,4,6-trimethoxystyrylsulfonyl)methyl)-2-methoxyphenylamino)-2-phenylacetic acid; (R)-(E)-2-(5-((2,4,6-trimethoxystyrylsulfonyl)methyl)-2-methoxyphenylamino)-2-phenylacetic acid; (racemic)-(E)-2-(5-((2,4,6-trimethoxystyrylsulfonyl)methyl)-2-methoxyphenylamino)-2-phenylacetic acid; (E)-2-(5-((2,4,6-trimethoxystyrylsulfonyl)methyl)-2-methoxyphenyl-amino)-2-methylpropanoic acid; (E)-1-(2-(4-bromobenzylsulfonyl)vinyl)-2,4-difluorobenzene; and pharmaceutically acceptable salts thereof.

According to certain embodiments of the invention, the compound according to Formula I comprises an isolated optical isomer, substantially free of the opposite enantiomer. According to one preferred sub-embodiment, the isolated optical isomer has the (R)— absolute configuration at the atom designated by *, and is substantially free of the (S)-enantiomer. According to another preferred sub-embodiment, the isolated optical isomer has the (S)— absolute configuration at the atom designated by *, and is substantially free of the (R)-enantiomer.

The invention is also directed to the use of a compound according to Formula I, or pharmaceutically acceptable salt thereof, for preparation of a medicament for (1) treating a kinase-dependent proliferative disorder in an individual, which disorder is resistant to treatment with an ATP-competitive kinase inhibitor, or (2) preventing or delaying, in an individual suffering from a kinase dependent proliferative disorder, the development of resistance to therapy including administration of an ATP-competitive kinase inhibitor.

### DEFINITIONS

The term “individual” includes human beings and non-human animals.

The expression “effective amount” in connection with the treatment of a patient suffering from a proliferative disorder, particularly a cancer, refers to the amount of a compound of Formula I that inhibits the growth of cells that are proliferating at an abnormally high rate or alternatively induces apoptosis of such cells, preferably cancer cells, resulting in a therapeutically useful and selective cytotoxic effect on proliferative cells when administered to a patient suffering from a proliferative disorder, particularly a cancer. The term “effective amount” is inclusive of amounts of a compound of Formula I that may be metabolized to an active metabolite in an amount that inhibits the growth of abnormally proliferative cells or induces apoptosis of such cells.

The expression “proliferative disorder” means a disorder wherein cells are made by the body at an atypically accelerated rate. The expression “kinase-dependent proliferative disorder” refers to a proliferative disorder wherein the abnormally high cell proliferation is driven by the expression of a protein kinase.

The term “tumor” means an abnormal growth of tissue that results from abnormal cell proliferation, and which serves no physiological function. A tumor may be a benign tumor, i.e., a tumor which does not invade surrounding tissue and which does not otherwise endanger the life of the individual. A tumor may be cancerous. A cancerous tumor is malignant, i.e., it tends to invade surrounding tissue and/or to metastasize, i.e., to spread to tissues in the body that are remote from the original tumor site.

The expression “kinase inhibitor” refers to an agent that acts to inhibit the kinase activity of a kinase.

The expression “ATP-competitive kinase inhibitor” means a kinase inhibitor that inhibits the kinase by competing with ATP for the ATP binding site on the kinase.

The term “alkyl”, by itself or as part of another substituent means, unless otherwise stated, a straight, branched or cyclic chain saturated hydrocarbon radical, including di- and multi-radicals, having the number of carbon atoms designated in an expression such as (Cₓ-C_{y})alkyl. The expression (Cₓ-C_{y})alkyl wherein x<y, represents an alkyl chain containing a minimum of x carbon atoms and a maximum of y carbon atoms. Examples include: methyl, ethyl, propyl, isopropyl, butyl, isobutyl, tert-butyl, pentyl, neopentyl, hexyl, cyclohexyl and cyclopropylmethyl. Preferred is (C₁-C₃)alkyl, particularly ethyl, methyl and isopropyl.

The term “cycloalkyl” refers to alkyl groups that contain at least one cyclic structure. Examples include cyclohexyl, cyclopentyl, norbornyl, adamantyl and cyclopropylmethyl. Preferred is (C₃-C₁₂)cycloalkyl, particularly cyclopentyl, norbornyl, and adamantyl.

The term “alkylene” refers to a divalent alkyl radical having the number of carbon atoms designated (i.e. (C₁-C₆) means —CH₂—; —CH₂CH₂— —CH₂CH₂CH₂CH₂—; —CH₂CH₂CH₂CH₂CH₂—; and —CH₂CH₂CH₂CH₂CH₂CH₂—, and also includes branched divalent structures such as, for example, —CH₂CH(CH₃)CH₂CH₂— and —CH(CH₃)CH(CH₃), and divalant cyclic structures such as, for example 1,3-cyclopentyl.

The term “arylene”, by itself or as part of another substituent means, unless otherwise stated, a divalent aryl radical. Preferred are divalent phenyl radicals, or “phenylene” groups, particularly 1,4-divalent phenyl radicals.

The term “heteroarylene”, by itself or as part of another substituent means, unless otherwise stated, a divalent heteroaryl radical. Preferred are five- or six-membered monocyclic heteroarylene. More preferred are heteroarylene moieties comprising divalent heteroaryl rings selected from the group consisting of pyridine, piperazine, pyrimidine, pyrazine, furan, thiophene, pyrrole, thiazole, imidazole and oxazole, such as, for example 2,5-divalent pyrrole, thiophene, furan, thiazole, oxazole, and imidazole.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₆)alkoxy, particularly ethoxy and methoxy.

The carbon chains in the alkyl and alkoxy groups which may occur in the compounds of the invention may be cyclic, straight or branched, with straight chain being preferred. The expression “(C₁-C₆)alkyl” thus extends to alkyl groups containing one, two, three, four, five or six carbons. The expression “(C₁-C₆)alkoxy” thus extends to alkoxy groups containing one, two, three, four, five or six carbons.

The term “hydrocarbyl” refers to any moiety comprising only hydrogen and carbon atoms. The term includes, for example, alkyl, alkenyl, alkynyl, aryl and benzyl groups. Preferred are (C₁-C₇)hydrocarbyl. More preferred are (C₁-C₆)alkyl and (C₃-C₁₂)cycloalkyl.

The term “heteroalkyl”, by itself or in combination with another term means, unless otherwise stated, a stable straight or branched chain radical consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

The terms “halo” or “halogen” by themselves or as part of another substituent mean, unless otherwise stated, a fluorine, chlorine, bromine, or iodine atom.

The term “aromatic” refers to a carbocycle or heterocycle having one or more polyunsaturated rings having aromatic character (4n+2) delocalized π (pi) electrons). The term “aromatic” is intended to include not only ring systems containing only carbon ring atoms but also systems containing one or more non-carbon atoms as ring atoms. Systems containing one or more non-carbon atoms may be known as “heteroaryl” or “heteroaromatic” systems. The term “aromatic” thus is deemed to include “aryl” and “heteroaryl” ring systems.

The term “aryl” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl, which may be substituted or unsubstituted. The aforementioned listing of aryl moieties is intended to be representative, not limiting.

The term “heterocycle” or “heterocyclyl” or “heterocyclic” by itself or as part of another substituent means, unless otherwise stated, an unsubstituted or substituted, stable, monocyclic or polycyclic heterocyclic ring system which consists of carbon atoms and at least one heteroatom selected from the group consisting of N, O and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

Heterocyclyl groups are inclusive of monocyclic and polycyclic heteroaryl groups and monocyclic and polycyclic groups that are not aromatic, such as saturated and partially saturated and monocyclic and polycyclic partially saturated monocyclic and polycyclic groups.

The term “heteroaryl” or “heteroaromatic” refers to a heterocycle having aromatic character, and includes both monocyclic heteroaryl groups and polycyclic heteroaryl groups. A polycyclic heteroaryl group may include one or more rings which are partially saturated.

Examples of monocyclic heteroaryl groups include: Pyridyl; pyrazinyl; pyrimidinyl, particularly 2- and 5-pyrimidyl; pyridazinyl; thienyl; furyl; pyrrolyl, particularly 2-pyrrolyl and 1-alkyl-2-pyrrolyl; imidazolyl, particularly 2-imidazolyl; thiazolyl, particularly 2-thiazolyl; oxazolyl, particularly 2-oxazolyl; pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl; and 1,3,4-oxadiazolyl.

Examples of monocyclic heterocycles that are not aromatic include saturated monocyclic groups such as: Aziridine, oxirane, thiirane, azetidine, oxetane, thietane, pyrrolidine, pyrroline, imidazoline, pyrazolidine, dioxolane, 1,4-dioxane, 1,3-dioxane, sulfolane, tetrahydrofuran, thiophane, piperazine, morpholine, thiomorpholine, tetrahydropyran, homopiperazine, homopiperidine, 1,3-dioxepane, hexamethyleneoxide and piperidine; and partially saturated monocyclic groups such as: 1,2,3,6-tetrahydropyridine, 1,4-dihydropyridine, 2,3-dihydrofuran, 2,5-dihydrofuran, 2,3-dihydropyran, 1,2-dihydrothiazole, 1,2-dihydrooxazole, 1,2-dihydroimidizole and 4,7-dihydro-1,3-dioxepin.

Examples of polycyclic heteroaryl groups include: indolyl, particularly 3-, 4-, 5-, 6- and 7-imidolyl, quinolyl, isoquinolyl, particularly 1- and 5-isoquinolyl, cinnolinyl, quinoxalinyl, particularly 2- and 5-quinoxalinyl, quinazolinyl, phthalazinyl, 1,8-naphthyridinyl, 1,4-benzodioxanyl, coumarin, benzofuryl, particularly 3-, 4-, 1,5-naphthyridinyl, 5-, 6- and 7-benzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, tetrahydroquinolyl; 1,2,3,4-tetrahydroisoquinolyl; dihydrocumarinyl; 2,3-dihydrobenzofuryl; 2,3-dihydrobenzothienyl, N-methyl-2-indolinyl; and indolinyl.

Examples of non-aromatic polycyclic heterocycles include: pyrrolizidinyl and quinolizidinyl.

The aforementioned listing of non-aromatic heterocyclic moieties and heteroaryl moieties is intended to be representative, not limiting.

Preferred heteroaryl groups are 2-, 3- and 4-pyridyl; pyrazinyl; 2- and 5-pyrimidinyl; 3-pyridazinyl; 2- and 3-thienyl; 2- and 3-furyl; pyrrolyl; particularly N-methylpyrrol-2-yl; 2-imidazolyl; 2-thiazolyl; 2-oxazolyl; pyrazolyl; particularly 3- and 5-pyrazolyl; isothiazolyl; 1,2,3-triazolyl; 1,2,4-triazolyl; 1,3,4-triazolyl; tetrazolyl, 1,2,3-thiadiazolyl; 1,2,3-oxadiazolyl; 1,3,4-thiadiazolyl and 1,3,4-oxadiazolyl; indolyl, particularly 2-, 3-, 4-, 5-, 6- and 7-indolyl; cinnolinyl; quinoxalinyl, particularly 2- and 5-quinoxalinyl; quinazolinyl, particularly 2-, 5-, 6-, 7- and 8-quinazolinyl; phthalazinyl; 1,8-naphthyridinyl; 1,5-naphthyridinyl, particularly 1,5-naphthyridin-3-yl and 1,5-naphthyridin-4-yl; 1,4-benzodioxanyl; coumarinyl; benzofuryl, particularly 2-, 3- 5-, 6- and 7-benzofuryl; 1,2-benzisoxazolyl; benzothienyl, particularly 2-, 3-, 4-, 5-, 6-, and 7-benzothienyl; benzoxazolyl; benzthiazolyl, particularly 2-benzothiazolyl and 5-benzothiazolyl; purinyl; benzimidazolyl, particularly 2-benzimidazolyl; benztriazolyl; thioxanthinyl; carbazolyl; carbolinyl; and acridinyl, particularly 6-acridinyl.

More preferred heteroaryl groups are 2, 3- and 4-pyridyl; 2- and 3-thienyl; 2- and 3-furyl; 2-pyrrolyl; 2-imidazolyl; 2-thiazolyl; 2-oxazolyl; 2- and 3-indolyl; 2-, and 3-benzofuryl; 3-(1,2-benzisoxazolyl); 2-, and 3-benzothienyl; 2-benzoxazolyl; 1- and 2-benzimidazolyl, 2-, 3- and 4-quinolyl; and 2- and 5-benzthiazolyl. Most preferred heteroaryl groups are 2- and 3-indolyl; 2- and 3-pyrrolyl, 2-, and 3-benzofuryl; and 2-, and 3-benzothienyl.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position.

The α,β-unsaturated (aryl or heteroaryl) sulfones, sulfoxides and sulfonamides are characterized by isomerism resulting from the presence of a double bond. This isomerism is commonly referred to as cis-trans isomerism, but the more comprehensive naming convention employs E and Z designations. The compounds are named according to the Cahn-Ingold-Prelog system, the IUPAC 1974 Recommendations, Section E: Stereochemistry, in Nomenclature of Organic Chemistry, John Wiley & Sons, Inc., New York, N.Y., 4^{th} ed., 1992, p. 127-138. Using this system of nomenclature, the four groups about a double bond are prioritized according to a series of rules. Then, that isomer with the two higher ranking groups on the same side of the double bond is designated Z (for the German word “zusammen”, meaning together). The other isomer, in which the two higher-ranking groups are on opposite sides of the double bond, is designated E (for the German word “entgegen”, which means “opposite”). Thus if the four groups on a carbon-carbon double bond are ranked with A being the lowest rank and D being highest, A>B>C>D, the isomers would be named as in Scheme 1.

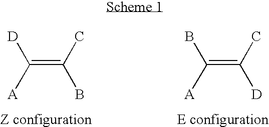

The present invention contemplates α,β-unsaturated (aryl or heteroaryl) sulfones, sulfoxides and sulfonamides in the E-configuration.

Some of the α,β-unsaturated (aryl or heteroaryl) sulfones, sulfoxides and sulfonamides according to Formula I may be characterized by isomerism resulting from the presence of a chiral center at X, when X is CH and R is other than —H. The isomers resulting from the presence of a chiral center comprise a pair of nonsuperimposable isomers that are called “enantiomers.” Single enantiomers of a pure compound are optically active, i.e., they are capable of rotating the plane of plane polarized light. Single enantiomers are designated according to the Cahn-Ingold-Prelog system. March, Advanced Organic Chemistry, 4th Ed., (1992), p. 109. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example shown in Scheme 2 below, the Cahn-Ingold-Prelog ranking is A>B>C>D. The lowest ranking atom, D is oriented away from the viewer.

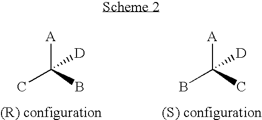

Unless otherwise indicated, both absolute configurations and mixtures thereof are included in the scope of α,β-unsaturated (aryl or heteroaryl) sulfones, sulfoxides and sulfonamides of Formula I.

The expression “substantially free” of the (R)- or (S)-enantiomer, when used to refer to an optically active compound according to Formula I, means the (R)- and (S)-enantiomers of the compound have been separated such that the composition is 80% or more by weight a single enantiomer. Preferably, the composition is 90% or more by weight a single enantiomer. More preferably, the composition is 95% or more by weight a single enantiomer. Most preferably, the composition is 99% or more by weight a single enantiomer.

Thus, by an (R)-enantiomer of a compound according to Formula I substantially free of the (S)-enantiomer” is meant the compound comprises 80% or more by weight of its (R)-enantiomer and likewise contains 20% or less of its (S)-enantiomer as a contaminant, by weight.

Isolated optical isomers may be purified from racemic mixtures by well-known chiral separation techniques. According to one such method, a racemic mixture of a compound having the structure of Formula I, or a chiral intermediate thereof, is separated into 99% wt. % pure optical isomers by HPLC using a suitable chiral column, such as a member of the series of DAICEL CHIRALPAK family of columns (Daicel Chemical Industries, Ltd., Tokyo, Japan). The column is operated according to the manufacturer's instructions.

For compounds according to Formula I, more than one chiral center may be present in a molecule. Two pairs of enantiomers result from the presence of two chiral centers. Only the relationship between the mirror-image isomers is termed enantiomeric. The relationship between a single enantiomer and other isomers that exist as a result of additional chiral centers is termed “diastereomeric.” Diastereomeric pairs may be resolved by known separation techniques including normal and reverse phase chromatography, and crystallization. Compounds of the present invention according to Formula I which contain two chiral centers, are understood to encompass isolated diastereomers, e.g., (R,R), (R,S), (S,R), and (S,S); isolated diastereomeric pairs, e.g., (R,R) and (R,S), or (S,R), and (S,S); and all mixtures of diastereomers in any proportions.

Nomenclature employed herein for providing systematic names for compounds disclosed herein may be derived using the computer program package, CHEMDRAW®, CambridgeSoft Corporation, Cambridge, Mass. 02140.

### DESCRIPTION OF THE FIGURES

FIG. 1 is a graph of the effect of Compound 1, imatinib or saline on in vivo growth of 32D/BCR-ABL^{T315}I cells in nude mice following intravenous injection of 1×10⁶ of the cells. Data is plotted as the average number of 32D/BCR-ABL^{T315I} cells per 10 fields+/−SEM (n=10).

FIG. 2 is a plot of the body weight of individual mice dosed with Compound 1 (100 mg/kg), imatinib (100 mg/kg) or saline. The weight of each mouse in the three dose groups was determined daily. The average body weights were plotted as a percent of starting body weight.

FIG. 3 is a graph of hematopoietic colony formation in CD-1 mice injected intravenously (tail vein injection) with saline or Compound 1 (100 mg/kg) dissolved in saline.

FIG. 4(a) is a plot of the percent viable CML K562 cells remaining after a 72 hour incubation with varying concentrations of Compound 1 or imatinib.

FIG. 4(b) is a plot of the percent viable murine 32Dcl3.BCR-ABL cells after a 72 hour incubation with varying concentrations of Compound 1 or imatinib.

FIG. 5(a) is a plot of the percent viable BCR-ABL T315L-expressing cells remaining after a 72 hour incubation with varying concentrations of Compound 1 or imatinib.

FIG. 5(b) is a plot of the percent viable BCR-ABL E255K-expressing cells remaining after a 72 hour incubation with varying concentrations of Compound 1 or imatinib.

FIG. 5(c) is a plot of the percent viable BCR-ABL Y253H-expressing cells after a 72 hour incubation with varying concentrations of Compound 1 or imatinib.

FIG. 5(d) is a plot of the percent viable BCR-ABL G250E-expressing cells after a 72 hour incubation with varying concentrations of Compound 1 or imatinib.

FIG. 6 is a graph of growth of 32Dcl3 cells transfected to express wild-type or imatinib-resistant forms of BCR-ABL and treated with Compound 2.

FIG. 7 is a plot of the percent viable cells expressing wild type BCR-ABL, or the BCR-ABL mutations G250E, T315I or M351T, after incubation with varying concentrations of Compound 4 for 72 hours.

### DETAILED DESCRIPTION OF THE INVENTION

According to the present invention, certain α,β-unsaturated sulfones sulfoxides and sulfonamides, or pharmaceutically acceptable salts thereof, are effective in inhibiting proliferation of target cells in kinase-dependent proliferative disorders that are resistant to treatment with ATP-competitive kinase inhibitors.

The compounds may be used in treating proliferative disorders in individuals whose disorder has become resistant to treatment with ATP-competitive kinase inhibitors due to mutations in the target kinase. For example, the compounds of the invention may be administered to treat imatinib-resistant CML and ALL, arising from point mutations in the target kinase, BCR-ABL. The compounds of the invention may be employed against other kinase-dependent proliferative disorders which have become refractory to therapy due to kinase mutations, such as proliferative disorders characterized by expression of KIT, PDGFRα, PDGFRβ, EGFR, SRC and P38. Mutations in these kinases have been shown to inhibit binding of imatinib and other ATP-competitive kinase inhibitors.

Proliferative disorders which may be driven by kinase expression include, for example, chronic myelogenous leukemia, acute lymphoblastic lymphoma, idiopathic pulmonary fibrosis, idiopathic hypereosinophilic syndrome, chronic myelomonocytic leukemia, malignant fibrous histiocytoma, prostate cancers, androgen dependent prostate cancers, dermatofibrosarcoma, endometrioid endometrial carcinoma, uterine papillary serous carcinoma, chordoma, glioma, malignant astrocytoma, glioblastoma, gastrointestinal stromal tumors, medulloblastoma, uterine leiomyosarcomas, and non-small-cell lung cancer.

### I. Identification of Proliferative Disorders Resistant to ATP-Competitive Kinase Inhibitors.

Mutations within the BCR-ABL kinase domain in imatinib-treated chronic myeloid leukemia (CML) are the main mechanism of acquired resistance to imatinib in CML. Likewise, mutations within the kinase domain of genes encoding for other kinases, e.g., PDGFR, KIT, EGFR, SRC and P38, have been implicated as a primary mechanism for acquired resistance to protein kinase inhibitors which have antiproliferative activity versus the unmutated kinases.

Intervention in cases of drug resistance, by employing the method of the present invention, may be carried out following detection of kinase mutations in cells of the subject, particularly neoplastic cells of the subject. Mutations may be detected, for example, by sequencing the subject's relevant kinase genes in target cells, such as neoplastic cells, and comparing the sequence against a databank of resistance-conferring conferring mutations. In addition to direct DNA sequencing, the following methods known to those skilled in the art may also be used to detect kinase mutations: Single-strand Conformation Polymorphism (SSCP); Denaturing Gradient Gel Electrophoresis (DGGE); Denaturing High-Performance Liquid Chromatography (DHPLC); Chemical Mismatch Cleavage (CMC); Enzyme Mismatch Cleavage (EMC); Heteroduplex analysis; and the use of DNA microarrays.

According to one embodiment of the invention, quantitative polymerase chain reaction (RQ-PCR) of BCR-ABL mRNA in imatinib-treated subjects may be used to detect patients at risk of resistance. A significant portion of imatinib-treated subjects displaying a two-fold or more increase in bcr-abl expression have detectable BCR-ABL mutations, indicating that such a rise in BCR-ABL may serve as a primary indicator to test patients for imatinib-deactivating BCR-ABL kinase domain mutations (Branford et al., Blood, 104, 2926-32 (2004)). Elevated BCR-ABL expression in the cells of a subject undergoing ATP-competitive kinase inhibitor therapy would suggest the need for mutation analysis to identify possible resistance-conferring BCR-ABL mutations, particularly in the BCR-ABL kinase domain.

### II. Administration of Compounds

The compounds may be administered by any route, including oral and parenteral administration. Parenteral administration includes, for example, intravenous, intramuscular, intraarterial, intraperitoneal, intranasal, rectal, intravaginal, intravesical (e.g., to the bladder), intradermal, topical or subcutaneous administration. Also contemplated within the scope of the invention is the instillation of drug in the body of the patient in a controlled formulation, with systemic or local release of the drug to occur at a later time. For example, the drug may localized in a depot for controlled release to the circulation, or for release to a local site of tumor growth.

One or more compounds of Formula I may be administered simultaneously, by the same or different routes, or at different times during treatment.

Where both a Formula I compound and an ATP-competitive kinase inhibitor are both administered to the patient, the preferred schedule and dose will be dictated by the preferred mode for the ATP-competitive kinase inhibitor. The course of treatment differs from individual to individual, and those of ordinary skill in the art can readily determine the appropriate dose and schedule of administration of the ATP-competitive kinase inhibitor in a given clinical situation.

The specific dose of Formula I compound to obtain therapeutic benefit for treatment of a proliferative disorder will, of course, be determined by the particular circumstances of the individual patient including, the size, weight, age and sex of the patient, the nature and stage of the proliferative disorder, the aggressiveness of the proliferative disorder, and the route of administration of the compound.

For example, a daily dosage of from about 0.01 to about 50 mg/kg/day may be utilized, more preferably from about 0.05 to about 25 mg/kg/day. Particularly preferred are doses from about 0.5 to about 10.0 mg/kg/day, for example, a dose of about 5.0 mg/kg/day. The dose may be given over multiple administrations, for example, two administrations of 2.5 mg/kg. Higher or lower doses are also contemplated.

When both a Formula I compound and an ATP-competitive kinase inhibitor are both administered to the patient, the routes of administration may be the same or different for the two drugs. The drugs may be administered on the same or different days. According to one embodiment, the drugs are administered on succeeding days. Administering the drugs on the same day encompasses simultaneous or virtually simultaneous administration, up to administration within an about 24 hour timeframe of each other.

Dosing regimens for leukemic proliferative diseases, most particularly for chronic myelogenous leukemia, may include the following.

For a CML patient in chronic phase who is imatinib-resistant due to a mutation in the kinase domain of the bcr-abl gene, treatment comprises a daily oral dose of a Formula I compound ranging from about 1.5 mg/kg/day to about 4 mg/kg/day. If the desired therapeutic result is not achieved after 3 months of treatment, the dose is increased in about 0.5 to 1.5 mg/kg/day increments with ongoing monitoring occurring every 3 months. Upon appearance of disease progression, the dose is increased in about 1.0 to 3.0 mg/kg/day increments until the desired therapeutic result is achieved.

For a CML patient in either accelerated phase or blast phase, treatment comprises a daily oral dose of a Formula I compound ranging from about 2.5 mg/kg/day to about 4.5 mg/kg/day. If the desired therapeutic result is not achieved after 3 months of treatment, the dose is increased in about 0.5 to 1.5 mg/kg/day increments with ongoing monitoring occurring every 3 months. Upon appearance of disease progression, the dose is increased in about 1.0 to 3.0 mg/kg/day increments until the desired therapeutic result is achieved.

For treatment of a CML patient who becomes imatinib-resistant during a course of treatment with imatinib at 400 mg daily, the imatinib dose is increased to 600 or 800 mg daily and a daily oral dose of a Formula I compound about 2.0 to 5.0 mg/kg/day is added. If the desired therapeutic result is not achieved after 3 months of treatment, the dose of the Formula I compound is increased in about 0.5 to 1.5 mg/kg/day increments with ongoing monitoring occurring every 3 months. Upon appearance of disease progression, the dose of the Formula I compound is increased in about 1.0 to 3.0 mg/kg/day increments until the desired therapeutic result is achieved.

As a first line therapy for CML patients, treatment comprises a combination of imatinib and a Formula I compound. A daily oral dose of 400 milligrams of imatinib and about 1.0 to 4.0 mg/kg/day of a Formula I compound is administered. If the desired therapeutic result is not achieved after 3 months of treatment, the dose of the Formula I compound is increased in about 0.5 to 1.5 mg/kg/day increments with ongoing monitoring occurring every 3 months. Upon disease progression, the imatinib dose is increased to 600 or 800 milligrams and the dose of the Formula I compound is increased in about 1.0 to 3.0 mg/kg/day increments until the desired therapeutic result is achieved.

In each of the regimens above, treatment is ongoing, assuming acceptable toxicity, until the patient expires or other factors, such as terminal stage disease, dictate cessation of the treatment. Monitoring frequency of disease status can be adjusted as necessary by one skilled in the art.

Monitoring of disease status is well known in the art. For monitoring of leukemic proliferative diseases, most particularly chronic myelogenous leukemia, disease markers include but are not limited to: blood counts, cytogenetic monitoring of bone marrow cells (e.g. percent of Philadelphia-positive metaphases) and molecular markers (e.g. amount of bcr-abl mRNA transcript measured for instance by RQ-PCR or molecular rearrangements by fluorescence in situ hybridization (FISH)).

### III. Pharmaceutical Compositions

The Formula I compounds may be administered in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent or excipient which is compatible with the other ingredients of the formulation and to deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorobutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

### IV. Salts of Compounds According to Formula I

The Formula I compounds may take the form of salts. The term “salts”, embraces addition salts of free acids or free bases which are compounds of the invention. The term “pharmaceutically-acceptable salt” refers to salts which possess toxicity profiles within a range that affords utility in pharmaceutical applications.

Suitable pharmaceutically-acceptable acid addition salts may be prepared from an inorganic acid or from an organic acid. Examples of inorganic acids include hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, salicyclic, 4-hydroxybenzoic, phenylacetic, mandelic, pamoic, methanesulfonic, ethanesulfonic, benzenesulfonic, pantothenic, trifluoromethanesulfonic, 2-hydroxyethanesulfonic, toluenesulfonic, sulfanilic, cyclohexylaminosulfonic, stearic, alginic, γ-hydroxybutyric, galactaric and galacturonic acid.

Suitable pharmaceutically-acceptable base addition salts of Formula I compounds include for example, metallic salts including alkali metal, alkaline earth metal and transition metal salts such as, for example, calcium, magnesium, potassium, sodium and zinc salts. Pharmaceutically-acceptable base addition salts also include organic salts made from basic amines such as, for example, N,N′-dibenzylethylenediamine, chloroprocaine, choline, diethanolamine, ethylenediamine, meglumine (N-methylglucamine) and procaine.

All of these salts may be prepared by conventional means from the corresponding compound according to Formula I by reacting, for example, the appropriate acid or base with a the compound according to Formula I.

### V. Preparation of α,β-Unsaturated Sulfones, Sulfoxides and Sulfonamides Useful in the Practice of the Invention

α,β-Unsaturated sulfones D1 (wherein Ar¹, Ar², R and * are as defined for Formula I compounds), may be prepared by Knoevenagel condensation of aromatic aldehydes with benzylsulfonyl acetic acids C1. The procedure is described by Reddy et al., Acta. Chem. Hung. 115:269-71 (1984); Reddy et al., Sulfur Letters 13:83-90 (1991); Reddy et al., Synthesis No. 4, 322-323 (1984); and Reddy et al, Sulfur Letters 7:43-48 (1987), the entire disclosures of which are incorporated herein by reference. The general synthesis according to a Knoevenagel condensation is depicted in Scheme 3 below.

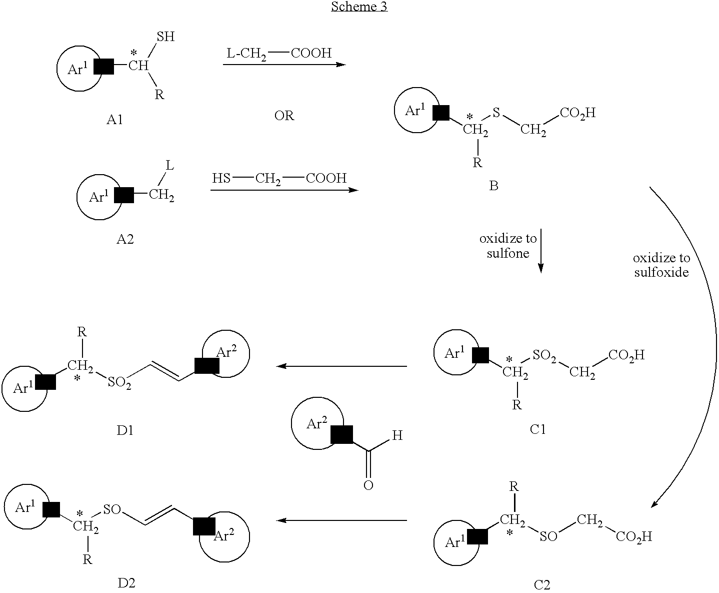

The starting benzylsulfonyl acetic acids C1, employed in Scheme 3 may be prepared by oxidation of the corresponding benzylmercaptoacetic acids B. The oxidation may be accomplished using any oxidation conditions suitable to oxidize a sulfide to a sulfone. Suitable reagents include peroxides such as hydrogen peroxide, peracids such as meta-chloroperoxybenzoic acid (MCPBA) and persulfates such as OXONE® (potassium peroxymonosulfate). The oxidation reaction is preferably carried out in the presence of a suitable solvent. Suitable solvents include, for example, water, acetic acid or non-polar solvents such as dichloromethane (DCM).

The benzylmercaptoacetic acids B may be prepared by reacting mercaptoacetic acid with a benzyl compound A2, or by reacting a haloacetic acid with a benzyl mercaptan A1.

As depicted in Scheme 3, α,β-unsaturated sulfoxides may be prepared by the Knoevenagel condensation of aromatic aldehydes with benzylsulfinyl acetic acids, C2.

The starting benzylsulfinyl acetic acids C2, employed in Scheme 3 in the preparation of sulfoxides D2, may be prepared by oxidation of the corresponding benzylmercaptoacetic acids B. The oxidation may be accomplished using any oxidation conditions suitable to oxidize a sulfide to a sulfoxide. Suitable reagents include peroxides such as hydrogen peroxide, peracids such as meta-chloroperoxybenzoic acid (MCPBA) and persulfates such as OXONE® (potassium peroxymonosulfate). The reaction is preferably carried out in the presence of a suitable solvent. Suitable solvents include, for example, water, acetic acid or non-polar solvents such as dichloromethane (DCM). The oxidation to a sulfoxide is generally performed at reduced temperature, e.g., from about -10° to about 25° C., and the oxidation reaction is monitored so as to terminate the reaction prior to over-oxidation to the sulfone.

α,β-Unsaturated sulfonamides G (wherein Ar¹, Ar², and R are as defined for Formula I), may be prepared by Knoevenagel condensation of aromatic aldehydes with arylaminosulfonylacetic acids F, as shown in Scheme 4.

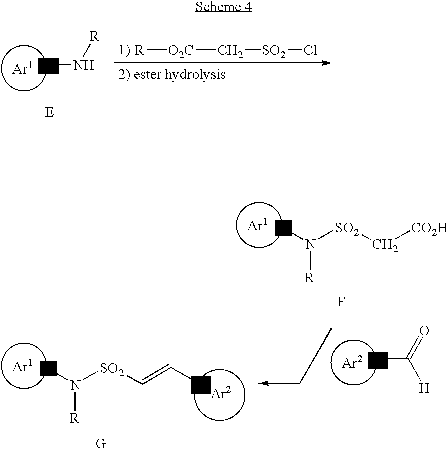

α,β-Unsaturated sulfonamides G, may alternately be prepared by reacting the aromatic amine E, with a aromatic sulfonylhalide H as depicted in Scheme 5. The aromatic sulfonylhalide H is preferably prepared by reacting an aromatic olefin, J with sulfuryl chloride.

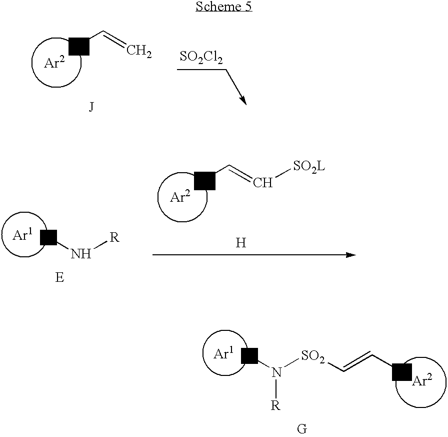

The preparation of compounds of Formula I is also described in one or more of the following US patents, the entire disclosures of which are incorporated herein by reference: U.S. Pat. Nos. 6,599,932, 6,576,675, 6,548,553, 6,541,475, 6,486,210, 6,414,034, 6,359,013, 6,201,154, 6,656,973 and 6,762,207.

The practice of the invention is illustrated by the following non-limiting examples. In the examples:

“Compound 1” is (E)-2-(5-((2,4,6-trimethoxystyrylsulfonyl)methyl)-2-methoxyphenylamino) propanoic acid:

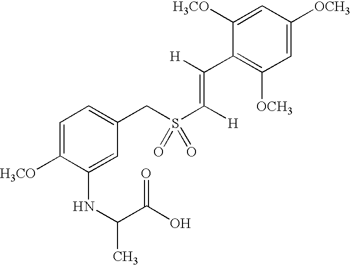

“Compound 2” is (E)-2-(5-((2,4,6-trimethoxystyrylsulfonyl)methyl)-2-methoxyphenylamino) acetic acid:

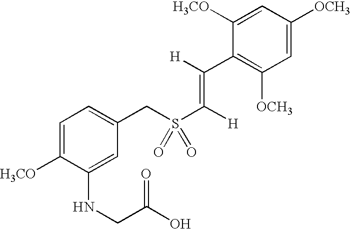

“Compound 3” is the sodium salt of (E)-2-(5-((2,4,6-trimethoxystyryl-sulfonyl)methyl)-2-methoxyphenylamino)-2-methylpropanoic acid:

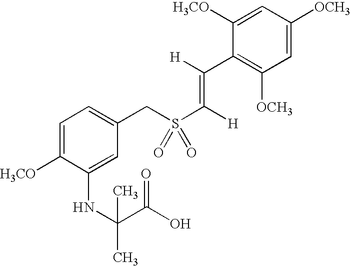

“Compound 4” is (E)-1-(2-(4-bromobenzylsulfonyl)vinyl)-2,4-difluorobenzene:

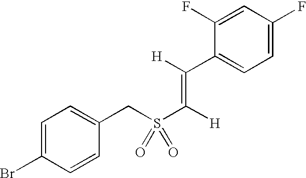

### EXAMPLES

### Example 1

### Effect of (E)-2-(5-((2,4,6-trimethoxystyrylsulfonyl)methyl)-2-methoxy-phenylamino)propanoic acid (Compound 1) on the In Vivo Growth of Cells Expressing the Imatinib Resistant BCR-ABL^{T315I}

A. 32Dcl3 Cell line

32Dcl3 Cells (Rovera et al., Oncogene, 1, 29-35 (1987)) were maintained in Iscove's Modified Dulbecco's Medium supplemented with 10% FBS, 1 U/mL penicillin-streptomycin and 10% WEHI-3B conditioned medium as a source of IL-3 (Ymer et al., Nature, 317, 255-258 (1985)).

### B. Generation of Wild-Type and Imatinib-Resistant Mutants of BCR-ABL.

Oligonucleotides corresponding to published bcr-abl mutations (Shah et al., Oncogene 22, 7389-85 (2003)) associated with imatinib resistance were used to introduce these mutations into the full-length bcr-abl cDNA using PCR-based site-directed mutagenesis (Myers et al., PCR Technology, eds. Erlich, H. A., Stockton Press, London (1989)). All constructs were verified by sequence analysis. pcDNA3-based expression plasmids encoding wild-type and imatinib resistant forms of BCR-ABL were introduced into actively proliferating 32Dc13 cells by electroporation as previously described (Kumar et al., Mol. Cell. Biol., 23, 663145 (2003)) and cells selected in the absence of IL-3. The expression of the BCR-ABL proteins was determined, as described below, by Western blot and kinase assays.

### C. Analysis of In Vivo Growth of T3151 Cells

Female athymic nude mice (ncr/ncr) were injected intravenously with 1×10⁶ 32Dcl3 cells expressing the mutant BCR-ABL^{T315I} via the tail vein. Treatments (3 groups with 10 mice per dosage group) were started 24 hours after cell injections by daily intraperitoneal injections of saline (vehicle), Compound 1 (100 mg/kg) or imatinib (100 mg/kg). Imatinib injections were terminated after 10 days due to toxicity.

(i) Toxicity of Imatinib and Compound 1

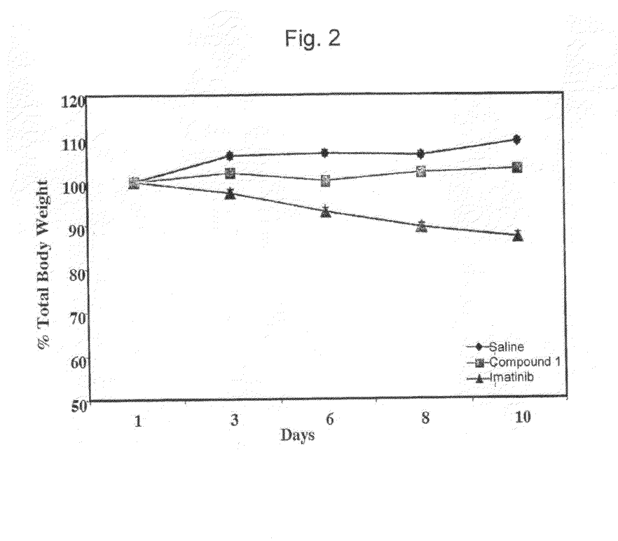

Body weights of the test animals were taken every two days. The body weight data is shown in FIG. 2. The Compound 1 treated mice showed no signs of toxicity, such as body weight loss, ruffled coats, lethargy or abnormal feces. In contrast, administration of imatinib for 10 days produced severe toxicity as judged by greater than a 20% loss of bodyweight, which resulted in the termination of drug administration.

(ii) Analysis of In Vivo Growth of T315I cells

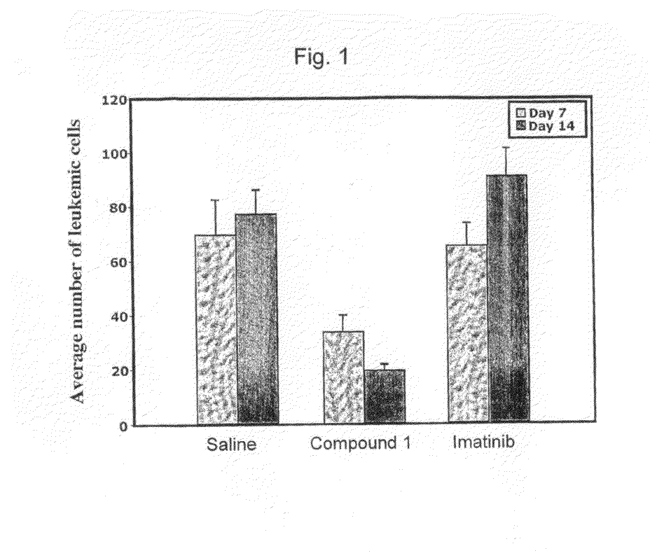

Blood smears on the treated mice were performed 7 and 14 days after the start of treatments. One drop of blood from each mouse was smeared onto glass slides, air dried and stained with modified Wright stain (Sigma). The T315I cells in the blood were easily visible due to their blue staining and size difference. The number of T315I cells per 10 fields was determined by counting 10 fields of view containing an equal density of red blood cells using a 40× objective on an upright Olympus microscope. The in vivo growth of T315I cells in the study are shown in FIG. 1. The number of T315I cells in the blood of mice treated with Compound 1 was significantly reduced on days 7 and 14 as compared to the number of cells found in the vehicle and imatinib treated groups.

### Example 2

### Hematopoietic Colony Formation Assay

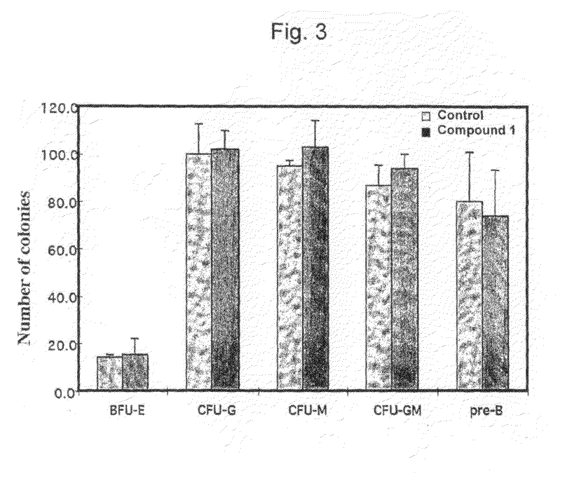

To examine the effects of Compound 1 on normal hematopoietic cell population in vivo, a dose of 100 mg/kg of this compound in normal saline (or saline alone as a control) was injected intravenously (tail vein injection) into CD-1 mice (10 animals per group). The effect on the in vitro hematopoietic colony formation of normal bone marrow cells derived from these mice was determined 24 hrs following administration. Bone marrow cells were extracted from the mice after 24 hrs and 2×10⁵ cells were plated on methylcellulose containing appropriate cytokines for lineage specific colony formation. Colonies were counted after 5 to 14 days of incubation. Data for the bone marrow colony formation assay is shown in FIG. 3. No reduction in colony formation of the erythroid, myeloid or lymphoid lineages with administration of Compound 1.

### Example 3

### Activity of Compound 1 Against BCR-ABL Expressing Cell Lines

A. Cell lines

K562 cells were maintained in RPMI (Roswell Park Memorial Institute) medium supplemented with 10% FBS and 1 U/ml penicillin-streptomycin.

32Dcl3 Cells (Rovera et al., Oncogene, 1, 29-35 (1987)) were maintained in Iscove's Modified Dulbecco's Medium supplemented with 10% FBS, 1 U/mL penicillin-streptomycin and 10% WEHI-3B conditioned medium as a source of IL-3 (Ymer et al., Nature, 317, 255-258 (1985)).

B. Cell Viability on Treatment with Imatinib or Compound 1

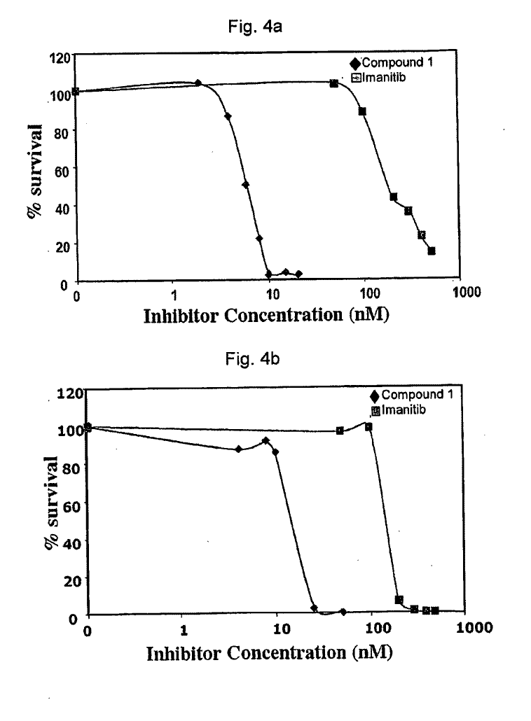

K562 cells, and murine 32Dcl3.BCR-ABL cells that ectopically expressed the wild-type p210^{BCR-ABL} oncoprotein, were incubated with varying concentrations of Compound 1 for a period of 72 hours. Cell viability was then determined by trypan blue exclusion. The percent viable cells compared to vehicle-treated controls was determined. In both cell lines, incubation with Compound 1 resulted in a rapid loss of viability with an LD₅₀ of 10-15 nM as shown in FIGS. 4a and 4b. The cell viability assay was also performed using imatinib. The IC₅₀ of imatinib was found to be greater than 100 nM (FIGS. 4a and 4b). The data for imatinib agrees with published data (O'Dwyer, Id.).

Both Compound 1 and imatinib inhibit proliferation of BCR-ABL⁺ cells. Compound 1 inhibited proliferation of BCR-ABL⁺ cell lines at a concentration 10-fold less than that shown for imatinib.

### Example 5

### Activity of Compound 1 Against Imatinib-resistant Tumor Cell Lines

Mutations corresponding to those published in the literature (Shah et al., Oncogene, 22, 7389-7395 (2003)) were introduced into the bcr-abl cDNA, which was then introduced into actively proliferating 32Dcl3 cells (Rovera et al., Oncogene 1, 29-35 (1987)) according to the following procedure.

Oligonucleotides corresponding to published bcr-abl mutations (Shah et al., Oncogene 22, 7389-85 (2003)) associated with imatinib resistance were used to introduce these mutations into the full-length bcr-abl cDNA using PCR-based site-directed mutagenesis (Myers et al., PCR Technology, eds. Erlich, H. A., Stockton Press, London (1989)). All constructs were verified by sequence analysis. pcDNA3-based expression plasmids encoding wild-type and imatinib resistant forms of BCR-ABL were introduced into actively proliferating 32Dcl3 cells by electroporation as previously described (Kumar et al., Mol. Cell. Biol., 23, 663145 (2003)) and cells selected in the absence of IL-3. The expression of the BCR-ABL proteins was determined by Western blot and kinase assays.

Transfectants were selected in the absence of IL-3. The expression and activity of each mutant BCR-ABL protein was confirmed by Western blot analysis and kinase assays (data not shown). All mutants conferred imatinib resistance at levels comparable to previously published studies when compared to wild-type p210^{BCR-ABL} expressing cells (data not shown) (Azam et al., Cell, 112, 831-843 (2003)).

The transfected cells were then cultured in the presence of various concentrations of Compound 1 or imatinib. The total number of viable cells was determined by trypan blue exclusion at 72 hours post-treatment. The percent viable cells compared to vehicle-treated controls was determined. All of the transfected cell lines, including cells expressing the T315I mutant, were found to be extremely sensitive to the growth inhibitory activity of Compound 1. The sensitivity of the cell lines, expressed in terms of GI₅₀ (the concentration which achieved 50% growth inhibition), is listed in Table 2 for 16 clinically relevant BCR-ABL mutations.

TABLE 2
Compound 1-induced Growth Inhibition of Cells

| Mutation | GI₅₀ (nM) | Mutation | LD₅₀ (nM) |
|---|---|---|---|
| None (wt) | 10 | G250E | 5.9 |
| F317L | 7.8 | Y253F | 6.8 |
| H396R | 6.8 | F311L | 6.8 |
| M351T | 7.8 | T315I | 7.5 |
| H396P | 7.9 | E255V | 7.8 |
| Y253H | 7.8 | Q252H | 7.0 |
| M244V | 7.8 | L387M | 7.1 |
| E355G | 7.8 | E255K | 8.4 |
| F359V | 7.1 | | |

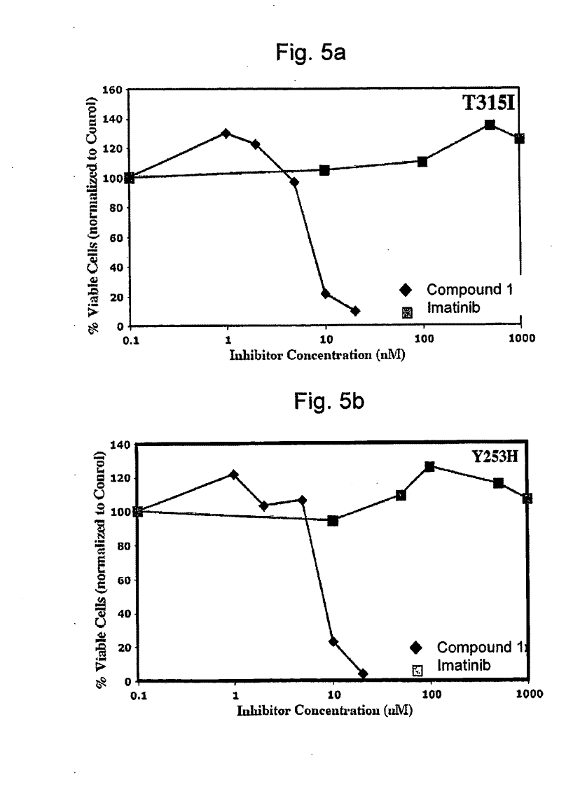

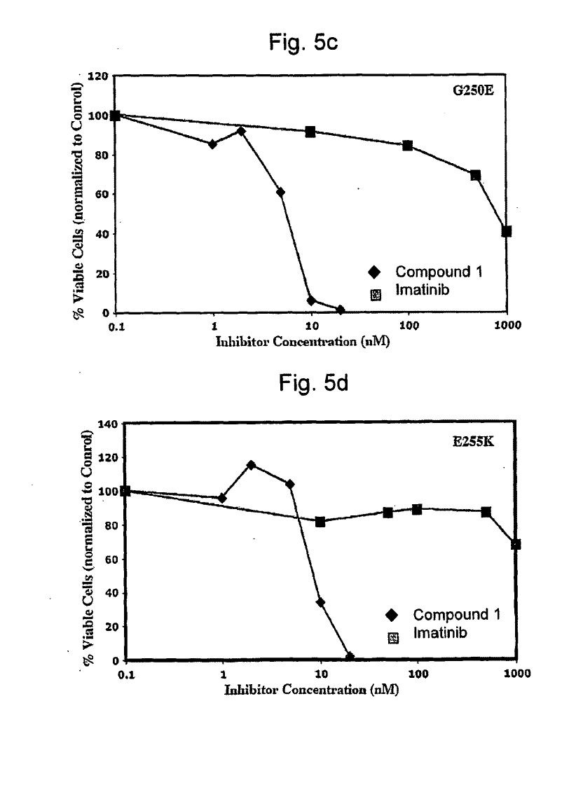

The result of four selected cell lines expressing BCR-ABL mutants T315I, E255K, Y253H and G250E are shown in FIGS. 5a, 5b, 5c and 5d, respectively.

### Example 6

### Activity of Compound 2 ((E)-2-(5-((2,4,6-trimethoxystyrylsulfonyl)methyl)-2-methoxyphenylamino)acetic acid) Against Imatinib-resistant Tumor Cell Lines

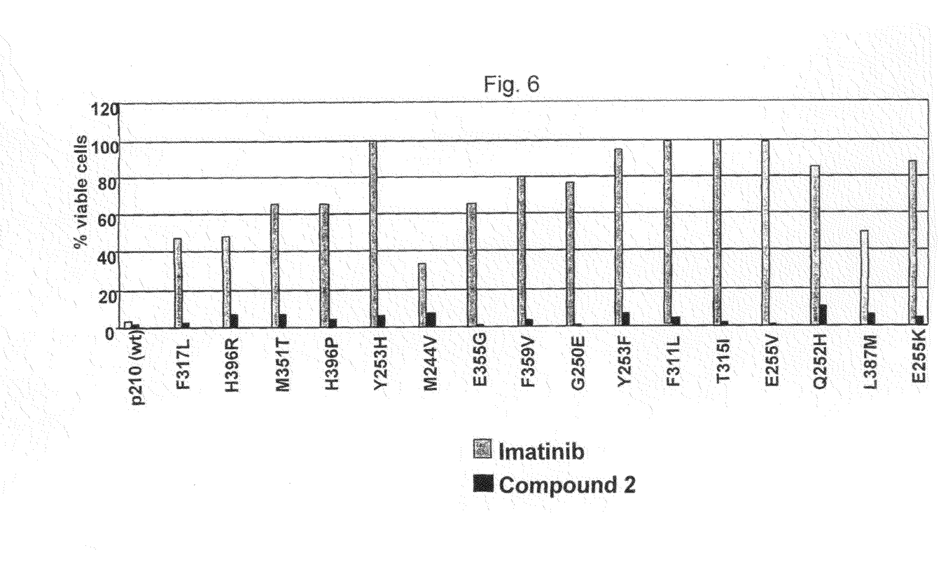

The cell lines listed in Table 2 were cultured in the presence of 250 nM Compound 2 or imatinib. The results are set forth in FIG. 6. All of the cell lines were sensitive to the growth-inhibitory activity of Compound 2.

### Example 7

### Treatment of Imatinib-Resistant CML in Chronic Phase

An adult patient diagnosed with chronic myelogenous leukemia is found to be imatinib-resistant. Sequencing of the bcr-abl gene expressed by leukemic cells confirms the presence of a mutation in the bcr-abl kinase domain. The patient's disease is in the chronic phase (fewer than 10% blasts, or 20% blasts and promyelocytes combined, in blood or bone marrow samples). The patient is treated with Compound 1 using the following dosing regimen: 200 milligrams of Compound 1 is administered orally once daily. Treatment is continued as long as there is no evidence of progressive disease or unacceptable toxicity. Upon appearance of disease progression to the accelerated phase (bone marrow or blood samples have more than 10% blasts (or 20% blasts and promyelocytes combined) but fewer than 30% blasts and promyelocytes), the daily dose of Compound 1 is increased to 300 milligrams. If necessary, additional dose increases are made, if no unacceptable toxicity occurs, to achieve the desired therapeutic result.

### Example 8

### Treatment of Imatinib-Resistant CML in Blast Phase

An adult patient diagnosed with chronic myelogenous leukemia is found to be imatinib-resistant. Sequencing of the bcr-abl gene expressed by leukemic cells confirms the presence of a mutation in the bcr-abl kinase domain. The patient's disease is in the blast phase. The blast phase (also known as acute phase or blast crisis) is characterized by bone marrow and/or blood samples have more than 30% blasts and promyelocytes. The patient is treated with Compound 1 using the following dosing regimen: 300 milligrams of Compound 1 is administered orally once daily. Treatment is continued as long as there is no evidence of progressive disease or unacceptable toxicity. Upon appearance of disease progression, the dose is increased to 400 milligrams, administered in a single dose or optionally, in two 200 milligrams doses daily. If necessary, additional dose increases are made, if no unacceptable toxicity occurs, to achieve the desired therapeutic result.

### Example 9

### Treatment of CML Following Initial Treatment with Imatinib

An adult patient diagnosed with chronic myelogenous leukemia is initially treated with imatinib. Over time, the patient's disease becomes imatinib-resistant. Sequencing of the bcr-abl gene expressed by leukemic cells confirms the presence of a mutation in the bcr-abl kinase domain. The patient's treatment regimen is changed to a combination regimen as follows: Imatinib is increased to the maximum dose tolerated by patient (800 milligrams) and is administered twice daily as 400 milligram doses. A 300 milligram dose of Compound 1 is also administered orally once daily. Treatment is continued as long as there is no evidence of progressive disease or unacceptable toxicity. Upon appearance of disease progression, the amount of Compound 1 is increased as necessary to achieve the desired therapeutic result.

### Example 10

### First-Line Treatment of CML with Two Drugs

An adult patient is newly diagnosed with chronic myelogenous leukemia. Sensitivity to imatinib is unknown. As a first line therapy, the patient is administered a combination of imatinib and Compound I using the following dosing regimen: A once daily oral dose comprising 400 milligrams of imatinib and 100 milligrams of Compound 1 is administered. Treatment is continued as long as there is no evidence of progressive disease or unacceptable toxicity. Upon appearance of disease progression, the dose of imatinib is increased to 600 milligrams and the dose of Compound 1 is increased to 200 milligrams. If necessary, additional dose increases, if no unacceptable toxicity occurs, in either or both imatinib (up to 800 milligrams) and Compound 1 are made.

### Example 11

### Activity of Compound 3 ((E)-2-(5-((2,4,6-trimethoxystyryl-sulfonyl)methyl)-2-methoxyphenylamino)-2-methylpropanoic acid sodium salt) Against Imatinib-resistant Tumor Cell Lines

The cell lines listed in Table 3 were cultured in the presence of various concentrations of Compound 3 ((E)-2-(5-((2,4,6-trimethoxystyrylsulfonyl)methyl)-2-methoxyphenylamino)-2-methylpropanoic acid sodium salt). The wild type comprises 32Dcl3.BCR-ABL cells that ectopically expressed the wild-type p210^{BCR-ABL} oncoprotein. The total number of viable cells was determined by trypan blue exclusion at 72 hours post-treatment. The percent viable cells compared to vehicle-treated controls was determined. The sensitivity of the cell lines, expressed in terms of GI₅₀, is listed in Table 3. All of the cell lines were sensitive to the growth-inhibitory activity of Compound 3.

TABLE 3
Compound 3-induced Growth Inhibition of Cells.

| Mutation | GI₅₀ (μM) |
|---|---|
| Wild-type | 0.04 |
| T315I | 0.06 |
| G250E | 0.025 |
| M351T | 0.06 |
| F317L | 0.06 |
| H396P | 0.05 |
| F311L | 0.07 |
| Y253F | 0.04 |
| L387M | 0.05 |
| Q252H | 0.04 |
| E255K | 0.05 |
| Y253F | 0.07 |
| Y253H | 0.03 |
| E355G | 0.05 |
| H396R | 0.06 |
| F359V | 0.07 |
| E255V | 0.05 |

### Example 12

### Activity of Compound 4 ((E)-1-(2-(4-Bromobenzylsulfonyl)vinyl)-2,4-difluorobenzene) Against Imatinib-resistant Tumor Cell Lines

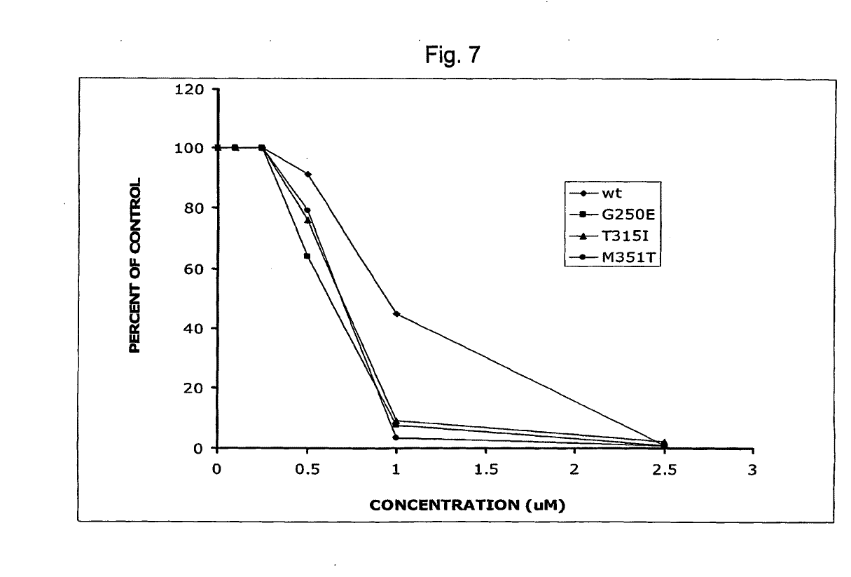

Cells expressing the wild-type p210^{BCA-ABL} oncoprotein (32Dcl3.BCR-ABL) or the BCR-ABL mutations G250E, T315I or M351T, were incubated with varying concentrations of Compound 4 ((E)-1-(2-(4-bromobenzylsulfonyl)vinyl)-2,4-difluorobenzene). The total number of viable cells was determined by trypan blue exclusion at 72 hours post-treatment. The percent viable cells compared to vehicle-treated controls was determined. The results are set forth in FIG. 7. All of the cell lines were sensitive to the growth-inhibitory activity of Compound 4.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

1. A method of treating a kinase-dependent proliferative disorder in an individual, which disorder is resistant to treatment with an ATP-competitive kinase inhibitor, said method comprising administering to the individual in need of such treatment an effective amount of at least one compound according to Formula I: 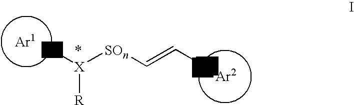
wherein:
Ar¹ and Ar² are independently selected from substituted and unsubstituted aryl and substituted and unsubstituted heteroaryl;
X is N or CH;
n is 1 or 2;
R is —H or —(C₁-C₈)hydrocarbyl; and
* indicates that, when X is CH, and R is other than —H, the conformation of the substituents on the carbon atom of X is R—, S— or any mixture of R— and S—; or a pharmaceutically acceptable salt thereof;
provided that when X is N, then n is 2.

2. The method according to claim 1, wherein substituents on substituted aryl and substituted heteroaryl Ar¹ are independently selected from the group consisting of halogen, —(C₁-C₈)hydrocarbyl, —C(═O)R², —NR²₂, —NHC(═O)R³, —NHSO₂R³, —NH(C₂-C₆)alkylene-C(═O)R⁶, —NHCR²R⁴C(═O)R⁶, —C(═O)OR², —C(═O)NR²₂, —NO₂, —CN, —OR², —OC(═O)R³, —OSO₂R³, —O(C₂-C₆)alkylene-C(═O)R⁶, —OCR²R⁴C(═O)R⁶, —P(═O)(OR²)₂, —OP(═O)(OR²)₂, —O(C₂-C₆ alkylene)N(C₁-C₃)alkyl)₂, —NHC(═NH)NHR², —(C₁-C₆)haloalkyl, —O(C₁-C₆)haloalkyl; —SO₂NH₂; and —N═CH—R⁷; and
substituents on substituted aryl and substituted heteroaryl Ar² are independently selected from the group consisting of —(C₁-C₈)hydrocarbyl, —C(═O)R², halogen, —NO₂, —CN, —OR², —C(═O)OR², —NR²₂, —(C₁-C₆)haloalkyl; —SO₂NH₂; and —O(C₁-C₆)haloalkyl; wherein:
each R² is independently selected from the group consisting of —H and —(C₁-C₈)hydrocarbyl;
each R³ is independently selected from the group consisting of —(C₁-C₈)hydrocarbyl, —O(C₁-C₈)hydrocarbyl, substituted and unsubstituted aryl, substituted and unsubstituted heterocyclyl(C₁-C₃)alkyl, substituted and unsubstituted heteroaryl(C₁-C₃)alkyl, —(C₂-C₁₀)heteroalkyl, —(C₁-C₆)haloalkyl, —CR²R⁴NHR⁵, —NR²₂, —(C₁-C₃)alkyleneNH₂, —(C₁-C₃)alkylene-N((C₁-C₃)alkyl)₂, —(C₁-C₃)perfluoroalkylene-N((C₁-C₃)alkyl)₂, —C₁-C₃)alkylene-N⁺((C₁-C₃)alkyl)₃, —(C₁-C₃)alkylene-N⁺(CH₂CH₂OH)₃, —(C₁-C₃)alkylene-OR², —(C₁-C₄)alkylene-CO₂R², —(C₁-C₄)alkylene-C(═O)halogen, halo(C₁-C₃)alkyl-, —(C₁-C₃)alkylene-C(═O)(C₁-C₃)alkyl, and —(C₁-C₄)perfluoroalkylene-CO₂R²;
each R⁴ is independently selected from the group consisting of —H, —(C₁-C₆)alkyl, —(CH₂)₃—NH—C(NH₂)(═NH), —CH₂C(═O)NH₂, —CH₂CO₂R², —CH₂SH, —(CH₂)₂C(═O)—NH₂, —(CH₂)₂CO₂R², —CH₂-(2-imidazolyl), —(CH₂)₄—NH₂, —(CH₂)₂—S—CH₃, phenyl, —CH₂-phenyl, —CH₂—OH, —CH(OH)—CH₃, —CH₂-(3-indolyl), and —CH₂-(4-hydroxyphenyl);
each R⁵ is independently selected from the group consisting of —H, —C(═O)(C₁-C₇)hydrocarbyl and a carboxy terminally-linked peptidyl residue containing from 1 to 3 amino acids in which the terminal amino group of the peptidyl residue is present as a functional group selected from the group consisting of —NH₂, —NHC(═O)(C₁-C₆)alkyl, —NH(C₁-C₆)alkyl, —N(C₁-C₆ alkyl)₂ and —NHC(═O)O(C₁-C₇)hydrocarbyl;
each R⁶ is independently selected from the group consisting of —OR², —NR²₂, and an amino terminally-linked peptidyl residue containing from 1 to 3 amino acids in which the terminal carboxyl group of the peptidyl residue is present as a functional group selected from the group consisting of —CO₂R² and —C(═O)NR²₂; and
each R⁷ is independently selected from the group consisting of substituted and unsubstituted aryl and substituted and unsubstituted heteroaryl;
provided that the highest number of substituents on Ar¹ and Ar² is equal to the number of substitutable hydrogen atoms in the ring to which the substituents are attached.

3. The method according to claim 2 wherein the compound according to Formula I is selected from the group consisting of: (E)-2-(5-((2,4,6-trimethoxystyrylsulfonyl)methyl)-2-methoxyphenylamino)acetic acid; (E)-(racemic)-2-(5-((2,4,6-trimethoxystyrylsulfonyl)methyl)-2-methoxyphenylamino)propanoic acid; (R)-(E)-2-(5-((2,4,6-trimethoxystyrylsulfonyl)methyl)-2-methoxyphenylamino)propanoic acid; (S)-(E)-2-(5-((2,4,6-trimethoxystyryl-sulfonyl)methyl)-2-methoxyphenylamino)propanoic acid; (E)-2-(5-((2,4,6-trimethoxystyrylsulfinyl)methyl)-2-methoxyphenylamino)acetic acid; (E)-2-(5-((2,4,6-trimethoxystyrylsulfinyl)methyl)-2-methoxyphenylamino)propanoic acid; (E)-2,4,6-trimethoxystyryl-N-(3-carboxymethylamino-4-methoxy-phenyl)sulfonamide; (S) (E)-2-(5-((2,4,6-trimethoxystyrylsulfonyl)methyl)-2-methoxyphenylamino)-2-phenylacetic acid; (R)-(E)-2-(5-((2,4,6-trimethoxystyrylsulfonyl)methyl)-2-methoxyphenylamino)-2-phenylacetic acid; (racemic)-(E)-2-(5-((2,4,6-trimethoxystyrylsulfonyl)methyl)-2-methoxyphenylamino)-2-phenylacetic acid; and pharmaceutically acceptable salts thereof.

4. The method according to claim 2 wherein the compound according to Formula I is selected from the group consisting of (E)-2-(5-((2,4,6-trimethoxystyryl-sulfonyl)methyl)-2-methoxyphenylamino)-2-methylpropanoic acid; (E)-1-(2-(4-bromobenzylsulfonyl)vinyl)-2,4-difluorobenzene; and pharmaceutically acceptable salts thereof.

5. The method according to claim 2 wherein the compound according to Formula I comprises an isolated optical isomer, substantially free of the opposite enantiomer.

6. The method according to claim 5 wherein the isolated optical isomer has the (R)-absolute configuration at the atom designated by *, and is substantially free of the (S)-enantiomer.

7. The method according to claim 5 wherein the isolated optical isomer has the (S)-absolute configuration at the atom designated by *, and is substantially free of the (R)-enantiomer.

8. The method according to claim 1, wherein the kinase-dependent proliferative disorder is resistant to treatment with at least one compound selected from the group consisting of PD180970, BMS-354825, imatinib, SU5416, SU6668, SU11248, AP23464, gefitinib, erlotinib, PD153035, and SB203580.

9. The method according to claim 1, wherein the proliferative disorder is resistant to treatment with an ATP-competitive inhibitor of BCR-ABL.

10. The method according to claim 9, wherein the resistance to treatment with the ATP-competitive BCR-ABL inhibitor results from a mutation of one or more amino acid residues of BCR-ABL.

11. The method according to claim 10, wherein the ATP-competitive inhibitor of BCR-ABL is imatinib.

12. The method according to claim 10, wherein the mutation comprises alteration of at least one amino acid residue within the BCR-ABL p-loop.

13. The method according to claim 12, wherein the mutation comprises an alteration of Tyr 253 or Glu255.

14. The method according to claim 10, wherein the mutation comprises alteration of at least one amino acid residue within the BCR-ABL activation loop.

15. The method according to claim 14, wherein the mutation comprises an alteration of His396.

16. The method according to claim 10, wherein the mutation comprises at least one mutation selected from the group consisting of F317L, H396R, M351T, H396P, Y253H, M244V, E355G, F359V, G250E, Y253F, F311L, T315I, E255V, Q252H, L387M, and E255K.

17. The method according to claim 1, wherein the proliferative disorder is selected from the group consisting of chronic myelogenous leukemia, acute lymphoblastic lymphoma, idiopathic pulmonary fibrosis, idiopathic hypereosinophilic syndrome, chronic myelomonocytic leukemia, malignant fibrous histiocytoma, prostate cancers, androgen dependent prostate cancers, dermatofibrosarcoma, endometrioid endometrial carcinoma, uterine papillary serous carcinoma, chordoma, glioma, malignant astrocytoma, glioblastoma, gastrointestinal stromal tumors, medulloblastoma, uterine leiomyosarcomas, and non-small-cell lung cancer.

18. The method according to claim 1, wherein the proliferative disorder is resistant to treatment with an ATP-competitive inhibitor of KIT.

19. The method according to claim 18, wherein the resistance to treatment with the ATP-competitive KIT inhibitor results from a mutation of one or more amino acid residues of KIT.

20. The method according to claim 19, wherein the mutation is selected from the group consisting T670I, Y823D and D816V.

21. The method according to claim 1, wherein the proliferative disorder is resistant to treatment with an ATP-competitive inhibitor of epidermal growth factor receptor.

22. The method according to claim 21, wherein the resistance to treatment with the ATP-competitive epidermal growth factor receptor inhibitor results from mutation of one or more amino acid residues of epidermal growth factor receptor.

23. The method according to claim 22, wherein the mutation comprises the mutation T766M.

24. The method according to claim 1, wherein the proliferative disorder is resistant to treatment with an ATP-competitive inhibitor of PDGFRα.

25. The method according to claim 24, wherein the resistance to treatment with the ATP-competitive inhibitor of PDGFRα results from mutation of one or more amino acid residues of PDGFRα.

26. The method according to claim 25, wherein the mutation comprises the mutation T674I.

27. The method according to claim 1, wherein the proliferative disorder is resistant to treatment with an ATP-competitive inhibitor of PDGFRβ.

28. The method according to claim 27, wherein the resistance to treatment with the ATP-competitive inhibitor of PDGFRβ results from mutation of one or more amino acid residues of PDGFRβ.

29. The method according to claim 28 wherein the mutation comprises T681I.

30. A method of preventing or delaying, in an individual suffering from a kinase dependent proliferative disorder, the development of resistance to therapy which includes administration of at least one ATP-competitive kinase inhibitor, said method comprising administering to an individual in need of such treatment an effective amount of at least one compound according to Formula I: 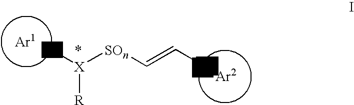
wherein:
Ar¹ and Ar² are independently selected from substituted and unsubstituted aryl and substituted and unsubstituted heteroaryl;
X is N or CH;
n is 0, 1 or 2;
R is —H or —(C₁-C₈)hydrocarbyl; and
* indicates that, when X is CH, and R is other than —H, the conformation of the substituents on the carbon atom of X is R—, S— or any mixture of R— and S—; or a pharmaceutically acceptable salt thereof;
provided that when X is N, then n is 2.

31. A method according to claim 30, further comprising administering an effective amount of the at least one ATP-competitive kinase inhibitor.

32. The method according to claim 31, wherein the at least one ATP-competitive kinase inhibitor is selected from the group consisting of PD180970, BMS-354825, imatinib, SU5416, SU6668, SU11248, AP23464, gefitinib, erlotinib, PD153035, and SB203580.
